(19) United States
(12) Patent Application Publication
(10) Pub. No.: US 2021/0267552 A1
(43) Pub. Date: Sep. 2, 2021

(54) SYSTEMS AND METHODS FOR DIGITALLY PROCESSING BIOPOTENTIAL SIGNALS

(71) Applicant: EasyG LLC, Beverly Hills, MI (US)

(72) Inventor: Alireza MOGHADDAMBAGHERI, Vancouver (CA)

(21) Appl. No.: 17/326,026

(22) Filed: May 20, 2021

Related U.S. Application Data
(63) Continuation of application No. PCT/US2019/063410, filed on Nov. 26, 2019.
(60) Provisional application No. 62/772,248, filed on Nov. 28, 2018.

(51) Int. Cl. A61B 5/00; A61B 5/282; A61B 5/318; A61B 5/0245; A61B 5/277

(52) U.S. Cl. CPC A61B 5/7207; A61B 5/282; A61B 5/318; A61B 5/398; A61B 5/0245; A61B 5/277; A61B 5/726

(57) ABSTRACT

Physiological parameter(s) are determined from a biopotential having one or more signal distorting elements. The method may involve suppressing one or more signal distorting elements may be from an acquired biopotential signal by decomposing the acquired biopotential signal, identifying the one or more signal distorting elements present in the acquired biopotential signal and reconstructing the decomposed biopotential signal without the one or more identified signal distorting elements. The method may involve determining a physiological parameter by analyzing decomposed elements of an acquired biopotential signal.

### CROSS-REFERENCE TO RELATED APPLICATIONS

This application is a continuation of Patent Cooperation Treaty (PCT) application No. PCT/US2019/063410 having an international filing date of 26 Nov. 2019 which in turn claims priority from, and the benefit under 35 U.S.C. § 119 in relation to, U.S. Application No. 62/772,248 filed 28 Nov. 2018. All of the applications referred to in this paragraph are hereby incorporated herein by reference for all purposes.

### TECHNICAL FIELD

The technology described herein relates to systems and methods for digitally processing a biopotential signal to determine useful physiological parameters. The technology described herein may suppress one or more signal distorting elements from electrocardiography (ECG) signals, electroencephalography (EEG) signals, electromyography (EMG) signals, electrooculography (FOG) signals and/or similar signals which represent physiological electrical activity at locations on, within, or proximate to, a subject's body.

### BACKGROUND

A conventional ECG system, for example, typically includes between 3 and 10 electrodes placed on areas of a subject's body to detect electrical activity of the subject's heart. The electrodes are connected to an ECG monitor by a commensurate number of wires/cables. A conventional ECG electrode typically comprises a resistive sensor element (i.e. a “contact” electrode) which is placed directly against the subject's skin. A number of electrodes are placed against the subject's skin to detect the electrical characteristics of the heart (e.g. the current through or voltage across the resistive sensor element) at desired vantage points on the subject's body. Alternatively, one or more electrodes may comprise a contactless sensor capacitively (or otherwise electrically) couplable to a tissue surface of the subject (i.e. a “contactless electrode”). The detected signals are relayed (typically through wires, but possibly wirelessly) to the ECG monitor, which is typically located on a lab table or the like, away from the subject's body. A signal processing unit within the ECG monitor processes the signals to generate an ECG waveform which can be displayed on a display of the ECG monitor.

FIGS. 1 and 2 show three electrodes 10, 12, 14 arranged in the so-called Einthoven's triangle on a subject's body 16. As is known in the art, electrodes 10, 12 and 14 may be respectively referred to as the Right Arm (RA), Left Arm (LA) and Left Leg (LL) electrodes because of the locations that they are commonly placed on body 16. To generate an ECG signal, various potential differences are determined between the signals from electrodes 10, 12, 14. These potential differences are referred to as “leads”. Leads have polarity and associated directionality. The common leads associated with the Einthoven's triangle shown in FIGS. 1 and 2 include: lead I (where the signal from RA electrode 10 is subtracted from the signal from LA electrode 12); lead II (where the signal from RA electrode 10 is subtracted from the signal from LL electrode 14); and lead III (where the signal from LA electrode 12 is subtracted from the signal from LL electrode 14).

In addition to the leads shown in FIG. 2, other common leads associated with the Einthoven's triangle configuration include: the AVR lead (where one half of the sum of the signals from LA and LL electrodes 12, 14 is subtracted from the signal for RA electrode 10); the ACL lead (where one half of the sum of the signals from RA and LL electrodes 10, 14 is subtracted from the signal for LA electrode 12); and the AVF lead (where one half of the sum of the signals from RA and LA electrodes 10, 12 is subtracted from the signal for LL electrode 14). As is known in the art, the AVR lead is oriented generally orthogonally to lead III, the AVL lead is oriented generally orthogonally to lead II and the AVG lead is oriented generally orthogonally to lead I. The signals from each of these leads can be used to produce an ECG waveform 18 as shown in FIG. 3. Additional sensors (e.g. electrodes) can be added to provide different leads which may be used to obtain different views of the heart activity. For example, as is well known in the art, sensors for precordial leads V1, V2, V3, V4, V5, V6 may be added and such precordial leads may be determined to obtain the so-called twelve-lead ECG.

Detected physiological electrical activity (e.g. electrical activity detected using, an ECG system, EEG system, EOG system, EMG system and/or the like) may, for example, also be used to determine non-electrical physiological parameters, such as, for example a respiratory rate of a subject.

However, one or more signal distorting elements (e.g. artifacts, noise, etc.) may mask and/or distort detected physiological electrical activity. In particular, signal distorting element(s) may distort biopotential signals which may comprise bioptential sensor signals from electrodes or other biopotential sensors or sensing circuits and/or biopotential signals (e.g. ECG leads) which are created (in the analog and/or digital domain) from combinations of biopotential sensor signals. For example, movement of a subject may result in electrical disturbances (i.e. motion artifacts) being introduced as inputs to circuitry (e.g. amplifier and/or signal processing circuitry) configured to receive biopotential sensor signals based on detected physiological electrical activity. Such electrical disturbances may also be introduced during detection of physiological electrical activity in non-stationary environments (e.g. moving vehicles, hospital beds, etc.). By way of non-limiting example, voluntary movements of a driver and/or passenger (e.g. pivoting of a steering wheel, feet movement, pressing one or more control pedals, shifting gears, head movement or the like), disturbances on a road surface (e.g. speed bumps, pot holes or the like), air turbulence, rough seas, etc. may result in one or more signal distorting elements (e.g. motion artifacts) being captured.

In some circumstances, one or more signal distorting elements may completely mask desired physiological electrical activity within a biopotential signal. For example, a signal distorting element may completely mask a QRS complex corresponding to a detected ECG signal.

Although conventional frequency based filtering techniques (low-pass filtering, high-pass filtering, band-pass filtering, etc.) are generally known in the art, such techniques are often not well-suited for suppressing one or more signal distorting elements from within biopotential signals. Typically, a signal distorting element (e.g. a motion artifact) comprises one or more frequencies falling within a frequency bandwidth corresponding to detected physiological electrical activity. In such circumstances, conventional frequency based filtering techniques cannot effectively suppress the one or more signal distorting elements without suppressing at least a portion of the detected electrical physiological activity.

There is a general desire for improved systems and methods for suppressing one or more signal distorting elements from biopotential signals, such as, by way of non-limiting example, biopotential signals associated with or corresponding to ECG, EEG, EMG and/or EOG signals. By way of non-limiting example, there is a general desire for systems and methods that can suppress a greater variety of signal distorting elements. There is also a general desire for systems and methods which may suppress one or more signal distorting elements while minimizing suppression of detected electrical physiological data.

There is also a general desire for systems and methods which can extrapolate physiological parameters (e.g. a respiratory rate of a subject) directly from noisy biopotential signals (i.e. biopotential signals having one or more signal distorting elements).

The foregoing examples of the related art and limitations related thereto are intended to be illustrative and not exclusive. Other limitations of the related art will become apparent to those of skill in the art upon a reading of the specification and a study of the drawings.

### SUMMARY

This invention has a number of aspects. These include, without limitation:
methods and apparatus for suppressing one or more signal distorting elements from an acquired biopotential signal;
methods and apparatus for digitally processing one or more acquired biopotential signals;
methods and apparatus for processing one or more acquired biopotential signals using Empirical Mode Decomposition;
methods and apparatus for processing one or more acquired biopotential signals using a Wavelet transform;
methods and apparatus for processing one or more acquired biopotential signals using an Independent Component Analysis;
methods and apparatus for extrapolating physiological parameters from noisy biopotential signals; and
biopotential measurement systems.

One aspect of the invention provides a method for suppressing one or more signal distorting elements (e.g. artifacts, noise, etc.) from an acquired biopotential signal. Such method includes acquiring a biopotential signal, converting the biopotential signal to a digital domain, digitally processing the biopotential signal to suppress one or more signal distorting elements and outputting the processed biopotential signal.

Another aspect of the invention provides a method for detecting the R-wave of a noisy ECG signal by analyzing the wavelet coefficients of a wavelet decomposition of the noisy ECG signal.

Another aspect of the invention provides a system for suppressing one or more signal distorting elements from a biopotential signal. Such system includes a plurality of electrode systems for acquiring the biopotential signal and a base unit for processing the biopotential signal. Each of the plurality of electrode systems may comprise a contact or contactless electrode and an amplifier circuit. Each of the plurality of electrode systems may be communicatively coupled to the base unit. The base unit may comprise a power supply, an I/O module and a processing module. In some embodiments, the processing module comprises a combining module, an analog to digital converter and a digital signal processing module. The digital signal processing module may be used to suppress one or more signal distorting elements from the biopotential signal.

Another aspect of the invention provides a method for determining a physiological parameter based on a biopotential signal indicative of a biopotential at a location on a body of a subject. The method involves acquiring a biopotential signal from the body of the subject using a plurality of electrodes. The acquired biopotential signal is converted to a digital signal. A wavelet decomposition is performed on the digital signal to generate a plurality of wavelet coefficients. The wavelet coefficients are analyzed to identify a time duration between local maximum values of some of the wavelet coefficients. Physiological parameters (e.g. heart rate) are determined based on the identified time duration.

Further aspects and example embodiments are illustrated in the accompanying drawings and/or described in the following description.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic illustration of the electrodes of a conventional ECG system arranged on the subject's body in an Einthoven's triangle configuration.

FIG. 2 is a schematic illustration of the electrodes of a conventional ECG system arranged in an Einthoven's triangle configuration and a number of corresponding leads.

FIG. 3 is a typical ECG waveform of the type that might be displayed on an ECG system.

FIG. 4 is a flow chart illustrating a method of determining a physiological parameter based on an acquired biopotential signal according to an example embodiment.

FIG. 5 is a flow chart illustrating a method for performing Empirical Mode Decomposition to process biopotential signal(s) according to an example embodiment.

FIG. 6A illustrates an example signal that may be decomposed using an example method described herein.

FIGS. 6B to 6F illustrate example Intrinsic Mode Functions corresponding to the FIG. 6A signal.

FIG. 6G illustrates an example processed signal corresponding to the FIG. 6A signal.

FIG. 6H illustrates an example unprocessed reconstructed signal corresponding to the FIG. 6A signal.

FIG. 7 is a flow chart illustrating a Wavelet method according to an example embodiment.

FIG. 7A is a schematic illustration showing an example Wavelet decomposition.

FIG. 7B is a schematic illustration showing an example Wavelet down-sampling.

FIG. 7C is a schematic illustration showing an example Wavelet up-sampling.

FIG. 7D is a schematic illustration showing an example Wavelet reconstruction.

FIG. 7E illustrates an example acquired biopotential signal. FIG. 7E also illustrates an example processed biopotential signal generated by processing the acquired biopotential signal using a Wavelet method according to an example embodiment.

FIGS. 7F to 7N illustrate example detail signals corresponding to an example nine-layer Wavelet decomposition.

FIG. 70 illustrates an example approximation signal corresponding to a ninth layer of an example nine-layer Wavelet decomposition.

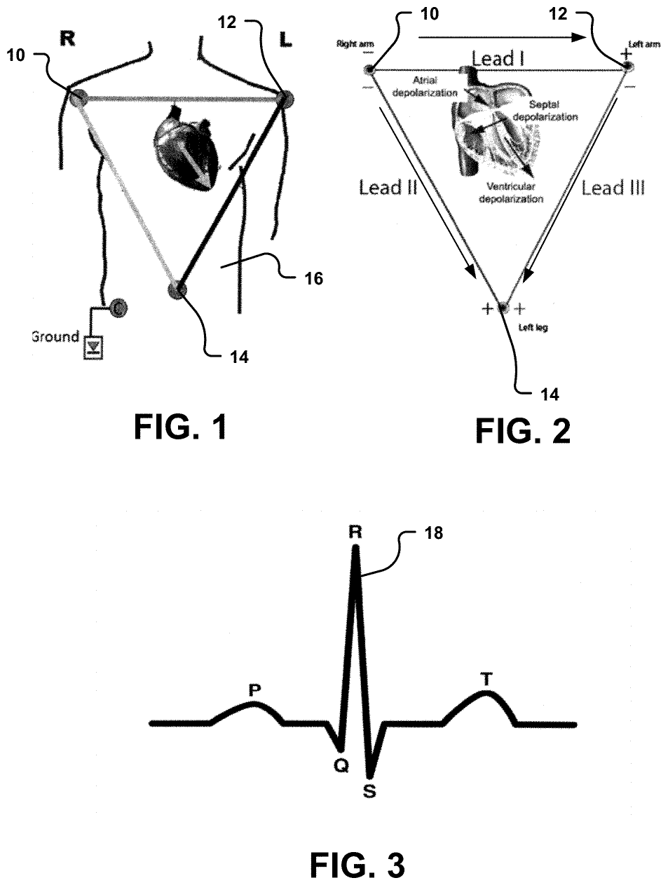

FIG. 8 is a flow chart illustrating a method for performing Independent Component Analysis to process biopotential signal(s) according to an example embodiment.

FIG. 8A is a flow chart illustrating a Fast Independent Component Analysis method according to an example embodiment.

FIG. 8B is a flow chart illustrating a real-time Independent Component Analysis method according to an example embodiment.

FIG. 9 is a schematic illustration of a biopotential measurement system according to an example embodiment.

FIGS. 9A and 9B are schematic illustrations of example electrode arrangements.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

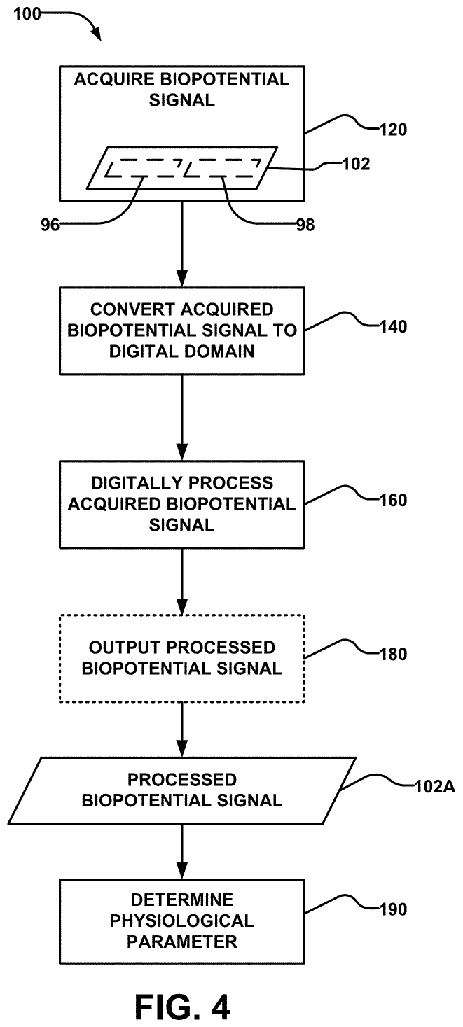

FIG. 4 is a flow chart illustrating an example method 100 for determining a physiological parameter based on an acquired biopotential signal 102 having one or more signal distorting elements (e.g. artifacts, noise, etc.) according to an exemplary embodiment. Biopotential signal 102 may, for example, be a signal representative of physiological electrical activity at locations on, within, or proximate to, a subject's body. Biopotential signal 102 may comprise a bioptential sensor signal from an electrode or other biopotential sensor or sensing circuit and/or a biopotential signal (e.g. an ECG lead) which is created (in the analog and/or digital domain) from a combination of biopotential sensor signals. By way of non-limiting example, biopotential signal 102 may be an ECG signal, EEG signal, EMG signal, EOG signal or the like. In some embodiments, method 100 completely removes one or more signal distorting elements from biopotential signal 102. In some embodiments, method 100 partially removes one or more signal distorting elements from biopotential signal 102.

Method 100 may, for example, be performed to suppress one or more motion artifacts present in biopotential signal 102 (i.e. method 100 may suppress signal distortions introduced into biopotential signal 102 as a result of movement of a subject during acquisition of biopotential signal 102 as described elsewhere herein). As described elsewhere herein, motion artifacts may frequently be present when, for example, biopotential signal 102 is acquired using contactless electrodes, biopotential signal 102 is acquired in a non-stationary environment (e.g. a moving vehicle, a hospital bed being moved from one care unit to another care unit, etc.) and/or the like. Alternatively, or in addition, method 100 may be performed to suppress artifacts such as, for example, loose electrode artifacts, wandering baseline artifacts, muscle tremor artifacts, breathing artifacts (i.e. artifacts resulting from a subject's breathing), human-induced artifacts (i.e. artifacts induced as a result of human interference with a subject such as, for example, performance of cardiopulmonary resuscitation (CPR) on the subject), neuromodulation artifacts, echo distortion artifacts, arterial pulse tapping artifacts and/or the like. Alternatively, or in addition, method 100 may suppress noise present in biopotential signal 102, such as noise arising from, for example, electromagnetic interference incident on at least one electrode used to acquire biopotential signal 102.

Method 100 commences in block 120 which comprises acquiring a biopotential signal 102. In currently preferred embodiments, biopotential signal 102 is acquired using a plurality of electrodes (or other sensors) coupled to a subject, although, in some embodiments, biopotential signal 102 could be acquired from a single sensor. The plurality of electrodes may comprise either contact and/or contactless electrodes. Each electrode in the plurality of electrodes generates an electrical signal corresponding to physiological electrical activity captured by a sensing portion of the electrode. Two or more electrical signals generated by the plurality of electrodes may be combined to generate biopotential signal 102 (e.g. a “lead” used in the ECG context). In some embodiments, a first electrode generates a reference signal 96 and a second electrode generates a data signal 98. Subtracting reference signal 96 from data signal 98 (and/or otherwise combining signals 96, 98) may, for example, generate biopotential signal 102.

Optionally, in some embodiments, block 120 may comprise conditioning one or more electrical signals generated by the plurality of electrodes prior to generating biopotential signal 102. For example, block 120 may amplify, filter, etc. one or more electrical signals generated by the plurality of electrodes. In some embodiments, amplifier gains, filter responses, etc. may be dynamically adjusted based on the one or more electrical signals generated by the plurality of electrodes to, for example, avoid amplifier saturation, dynamically filter the one or more electrical signals and/or the like. In some embodiments, block 120 generates a biopotential signal 102 before proceeding to condition (e.g. amplify, filter, etc.) biopotential signal 102.

In some embodiments, block 120 comprises receiving biopotential signal 102 in real time or in near-real time. In some embodiments, block 120 receives a pre-recorded biopotential signal 102. By way of non-limiting example, block 120 may receive pre-recorded biopotential signal 102 by block 120 retrieving pre-recorded biopotential signal 102 from memory, pre-recorded bipotential signal 102 being communicated to block 120 using a suitable network interface, a user inputting pre-recorded biopotential signal 102 and/or the like.

In some embodiments, block 120 may acquire a plurality of biopotential signals 102 (e.g. using a multi-lead system, such as a multi-lead ECG system). In such embodiments, each of the plurality of biopotential signals 102 may be processed using method 100. In some embodiments, each of the plurality of biopotential signals 102 is processed simultaneously. In some embodiments, each of the plurality of biopotential signals 102 is processed consecutively (i.e. one after the other).

Once biopotential signal 102 has been acquired, method 100 proceeds to block 140 which comprises converting acquired biopotential signal 102 to a digital domain (i.e. biopotential signal 102 is digitized). Biopotential signal 102 may be digitized using any known method of converting an analog signal to a digital signal. For example, as described elsewhere herein, a commercially available analog to digital converter may be used. In some embodiments, block 140 may dynamically adjust a digitization resolution on the basis of how biopotential signal 102 may be used in post-processing activity (e.g. a lower resolution may be required for an ECG signal that will be analyzed to determine a heart rate of a subject compared to an ECG signal that will be analyzed to determine whether any arrhythmias are present). Upon biopotential signal 102 being digitized, method 100 proceeds to block 160.

In block 160, biopotential signal 102 is digitally processed. In some embodiments, biopotential signal 102 is digitally processed to suppress one or more signal distorting elements that may be present in biopotential signal 102. As described elsewhere herein, block 160 may partially or completely remove one or more signal distorting elements that may be present in biopotential signal 102. In some embodiments, biopotential signal 102 comprises artifacts and/or noise having a spectral band that is distinct from a spectral band corresponding to detected electrical physiological data represented by biopotential signal 102. In such embodiments, block 160 may, for example, use one or more relatively simple frequency domain digital signal processing techniques, such as Fast Fourier Transform, Inverse Fast Fourier Transform, Short Time Fourier Transform and/or the like together with suitable frequency domain filtering techniques to suppress such artifacts and/or noise from biopotential signal 102.

In some embodiments, biopotential signal 102 comprises artifacts such as breathing artifacts. In some such embodiments, block 160 may perform a moving average technique to suppress artifacts from biopotential signal 102. In some embodiments, such moving average technique comprises a Zero Lag Exponential Moving Average (ZLEMA). Advantageously, a ZLEMA technique does not introduce a time lag into biopotential signal 102 (i.e. processed biopotential signal 102A is not time-shifted relative to acquired biopotential signal 102).

More complex artifacts and/or noise, such as, for example, motion artifacts, may have non-stationary properties with variable time-frequency attributes due to, for example, such artifacts and/or noise resulting from aperiodic movement of a subject during acquisition of biopotential signal 102. Suppression of such artifacts and/or noise in block 160 may involve a time-frequency analysis of biopotential signal 102. A suitable time-frequency analysis may comprise performing, for example, an Empirical Mode Decomposition method, a Wavelet method, an Independent Component Analysis method and/or the like. In some embodiments, the most computationally efficient time-frequency analysis is performed.

Empirical Mode Decomposition (EMD) comprises decomposing biopotential signal 102 into a plurality of so-called “Intrinsic Mode Functions” (IMFs), where the sum of the IMFs reconstruct decomposed biopotential signal 102. In particular, EMD may iteratively parse biopotential signal 102 into a plurality of “fast oscillation” and “slow oscillation” components (each component corresponding to a different IMF). Upon biopotential signal 102 being parsed into a plurality of IMFs, one or more IMFs corresponding to (e.g. comprising or otherwise corresponding to) artifacts and/or noise may be identified. Identified artifacts and/or noise may, for example, be suppressed by reconstructing biopotential signal 102 using only IMFs not identified as corresponding to artifacts and/or noise (i.e. any IMFs identified as corresponding to artifacts and/or noise are excluded during reconstruction of biopotential signal 102).

In currently preferred embodiments of EMD, each IMF comprises an equal number of extrema and zero-crossings. Each EMD may also be symmetric with respect to a local mean. In some embodiments, each IMF represents an oscillatory component of acquired biopotential signal 102.

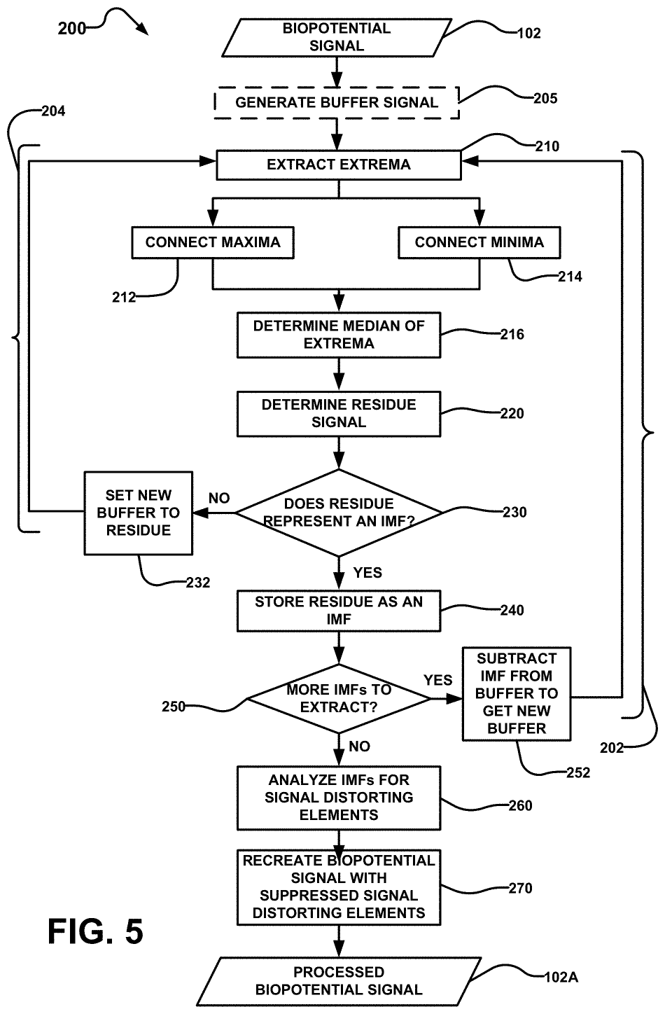

FIG. 5 is a flow chart illustrating an example method 200 for performing EMD to suppress one or more signal distorting elements from acquired biopotential signal 102.

Method 200 optionally commences in optional block 205 which comprises generating a buffer signal v to be processed by method 200. Buffer signal v may be generated by reproducing acquired biopotential signal 102. Advantageously, in embodiments involving the use of a buffer signal v, buffer signal v may be processed while preserving acquired biopotential signal 102 in its original state. Biopotential signal 102 (x[k]) may, for example, be represented as:

x[k]=Σₖ₌₁^{K}aₖ(t)ψₖ(t) (1)

where aₖ(t) represents “amplitude modulations” and ψₖ(t) represent “oscillations” inherent in biopotential signal 102. Buffer signal v may, for example, be represented as:

v[k]=x[k] (2).

In some embodiments, method 200 is performed using biopotential signal 102 directly. In the discussion that follows, it is assumed, without loss of generality that method 200 is performed on buffer signal v.

Upon buffer signal v being generated (or alternatively biopotential signal 102 being used directly), method 200 proceeds to IMF extraction loop 202. IMF extraction loop 202 iteratively extracts one or more IMFs corresponding to buffer signal v. IMF extraction loop 202 commences with sifting loop 204 which generates an IMF from buffer signal v.

Sifting loop 204 commences in block 210 which comprises extracting extrema (i.e. maxima and minima) from buffer signal v. Once extrema are extracted from buffer signal v, sifting loop 204 proceeds to blocks 212 and 214. In block 212, a line connecting the extracted maxima is generated. In block 214, a line connecting the extracted minima is generated. Block 214 may be performed simultaneously with block 212, before block 212 or after block 212. In some embodiments, a cubic spline method of interpolation (or some other suitable interpolation techniques) is used by block 212 and/or 214 to generate each line connecting the extracted maxima and minima respectively.

In block 216, sifting loop 204 proceeds to generate a mean m of upper and lower envelopes of the extracted extrema. Upon mean m being generated, sifting loop 204 proceeds to block 220 which comprises determining a residue h. Residue h may be determined by subtracting mean m from buffer signal v. For example, residue h may be represented as:

h=v−m (3).

Upon residue h being determined, sifting loop 204 proceeds to block 230 which comprises determining whether residue h corresponds to an IMF of buffer signal v.

In some embodiments, block 230 determines that residue h represents an IMF if the squared difference between two consecutive iterations of sifting loop 204 is smaller than a threshold value. For example, the squared difference SD may be represented as:

$$S {D}_{k} = \frac{{\sum }_{t = 0}^{T} {\vert {h}_{k - 1} ( t ) - {h}_{k} ( t ) \vert}^{2}}{{\sum }_{t = 0}^{T} {h}_{k - 1}^{2}} . \qquad ( 4 )$$

However, in some embodiments, the difference between two consecutive iterations of sifting loop 204 may be small even if residue h does not have an equal number of extrema and zero-crossings (i.e. residue h does not satisfy the definition of an IMF). Consequently, block 230 may also involve an inquiry into whether h satisfies the definition of an IMF.

Alternatively, or in addition, block 230 may determine that residue h represents an IMF using an S-number criterion. In such embodiments, sifting loop 204 will stop iterating after S consecutive iterations, where a number of zero-crossings and extrema of residue h stay the same and are equal or differ at most by one. In some embodiments, S-numbers between 4 and 8 may be used. In some embodiments, S-numbers between 3 and 5 may be used. In some embodiments, S-numbers may be used as described by Huang et al. (2003) in “A confidence limit for the empirical mode decomposition and Hilbert spectral analysis”, DOI: 10.1098/rspa.2003.1123, which is hereby incorporated herein by reference for all purposes. As with the SD, block 230 may also involve an inquiry into whether h satisfies the definition of an IMF.

If block 230 determines that residue h does not represent an IMF (e.g. new extrema were found), sifting loop 204 proceeds to block 232 which comprises setting a new buffer signal v to be residue h. Upon setting buffer signal v to residue h, block 232 returns sifting loop 204 to block 210. Alternatively, if block 230 determines that residue h represents an IMF, sifting loop 204 ends and IMF extraction loop 202 proceeds to block 240 which comprises storing residue h as an IMF.

Once an IMF is stored, IMF extraction loop 202 proceeds to block 250 which determines if a further IMF can be extracted from buffer signal v. In some embodiments, IMF extraction loop 202 is stopped if buffer signal v becomes smaller than a predetermined value and/or buffer signal v becomes a monotonic function from which no more IMFs can be extracted. If one or more further IMFs are to be extracted from buffer signal v, IMF extraction loop proceeds to block 252 which comprises subtracting residue h from buffer signal v to create a new buffer signal v before returning new buffer signal v to block 210 for further processing. If no further IMFs are to be extracted, IMF extraction loop 202 stops and method 200 proceeds to block 260.

In block 260, each IMF stored in block 240 is analyzed. For example, each IMF may be labelled as corresponding to (e.g. comprising) artifacts and/or noise. In some embodiments, each IMF is compared to sample IMFs known to comprise artifacts and/or noise. In some embodiments, amplitude and/or frequency values of each IMF are compared to threshold values known to correspond to artifacts and/or noise (e.g. a frequency value of 60 Hz may, for example, correspond to AC noise).

Upon each stored IMF being analyzed, method 200 proceeds to block 270 which comprises generating a processed biopotential signal 102A with one or more signal distorting elements being suppressed. Processed biopotential signal 102A (y(t)) may, for example, be represented as:

y(t)=rₙ+Σcₛ (5)

where rₙ represents a residue which can be either the mean trend or a constant, and cₛ represents the clean IMFs of the signal (i.e. IMFs identified as comprising no signal distorting elements). In some embodiments, where for example rₙ is representative of a DC component of biopotential signal 102, rₙ may also be removed from processed biopotential signal 102A.

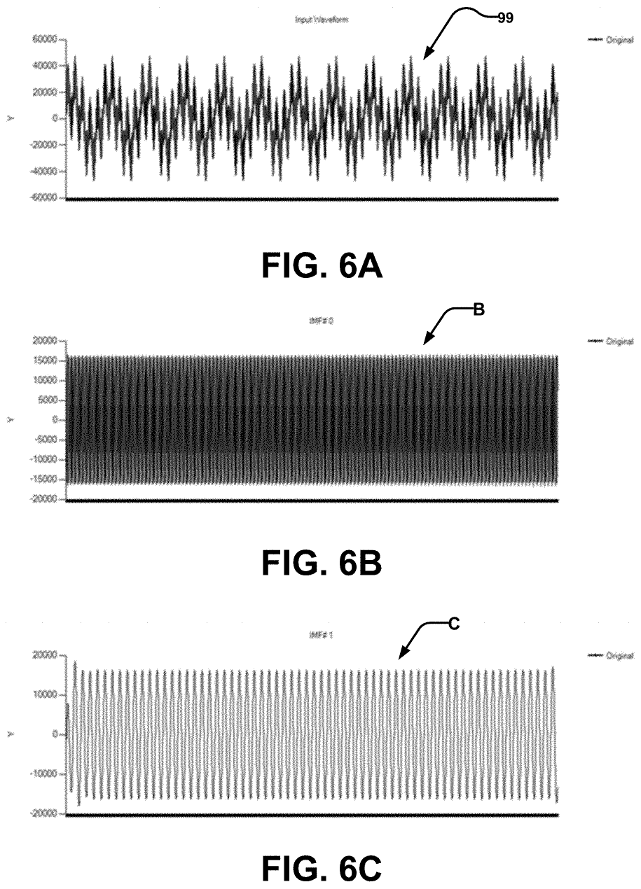

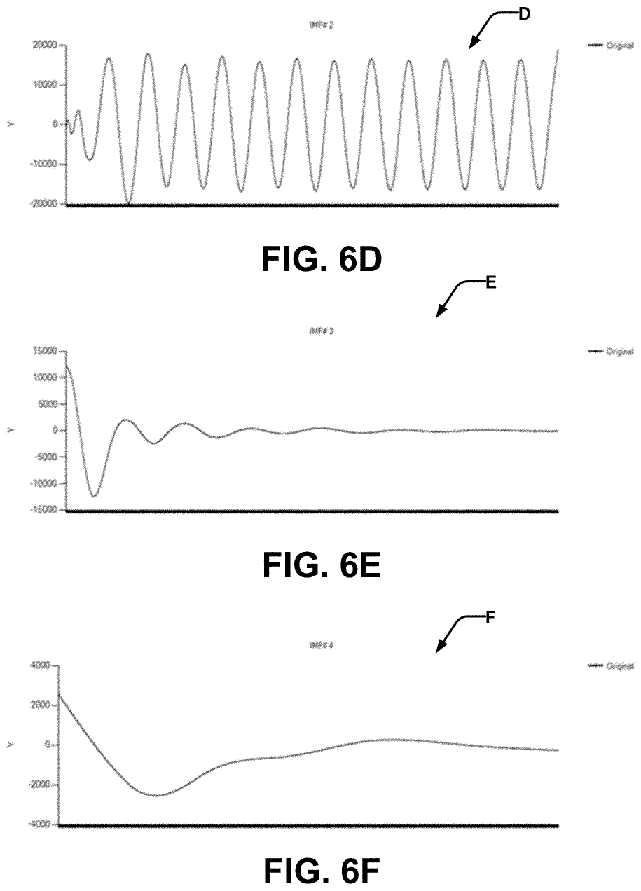

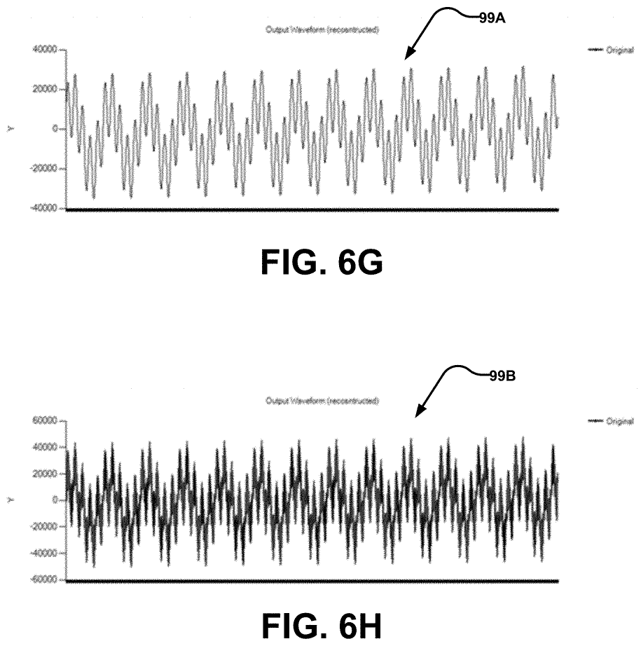

FIG. 6A illustrates an example complex signal 99. Signal 99 illustrated in FIG. 6A comprises a 2 Hz sine wave with a phase of 22 degrees, a 10 Hz sine wave with zero phase and a 60 Hz sine wave with a phase of 14 degrees. FIGS. 6B to 6F illustrate signal 99 decomposed into IMFs B to F respectively using, for example, method 200 described elsewhere herein. Method 200, may for example, identify IMF B illustrated in FIG. 6B as corresponding to 60 Hz AC noise. FIG. 6G illustrates an example processed signal 99A suppressing IMF B. FIG. 6H illustrates an example processed signal 99B reconstructed using all of IMFs B to F (e.g. no signal distorting elements have been suppressed).

In some embodiments, EMD comprises Complete Ensemble Empirical Mode Decomposition with Adaptive Noise. In some embodiments, EMD comprises Extended (or Ensemble) Empirical Mode Decomposition.

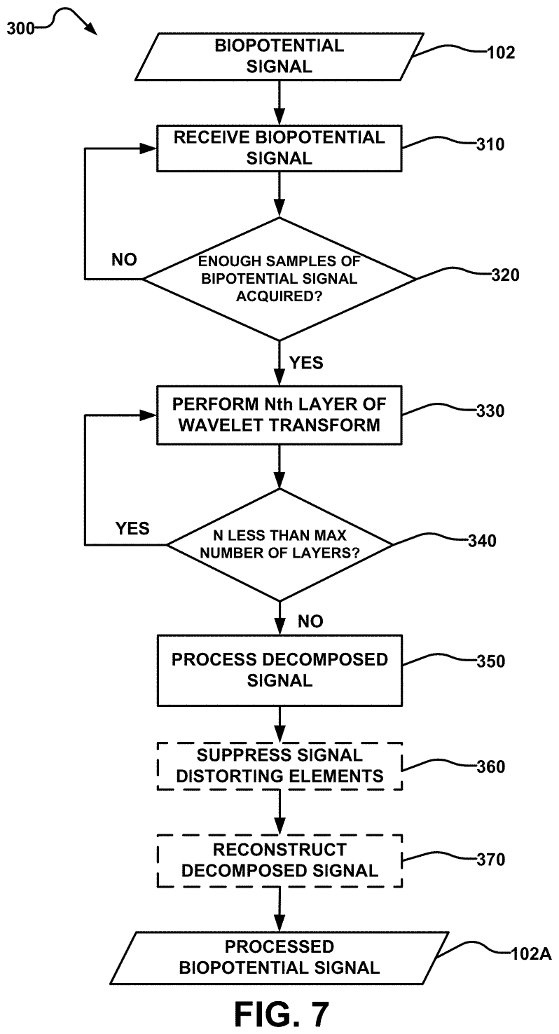

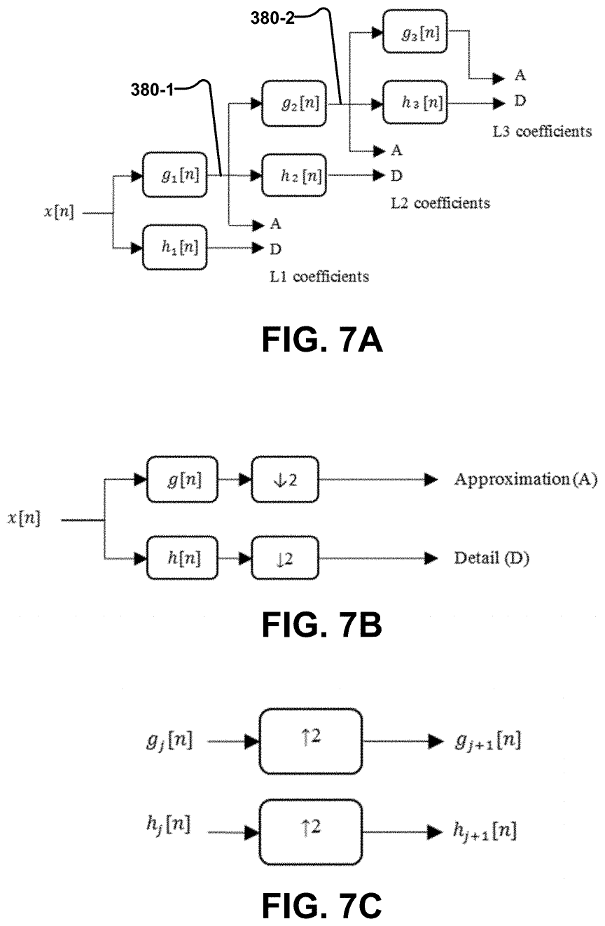

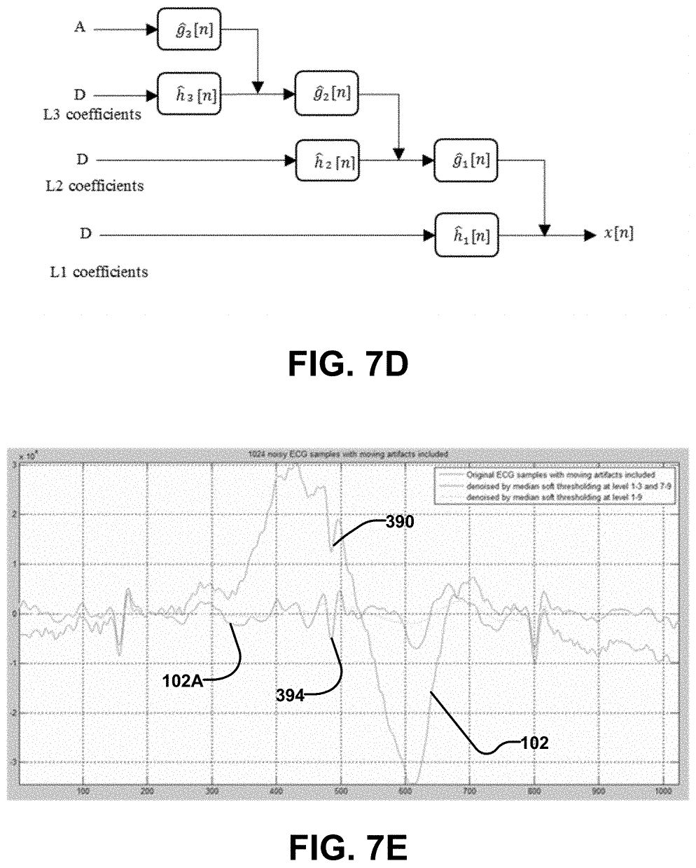

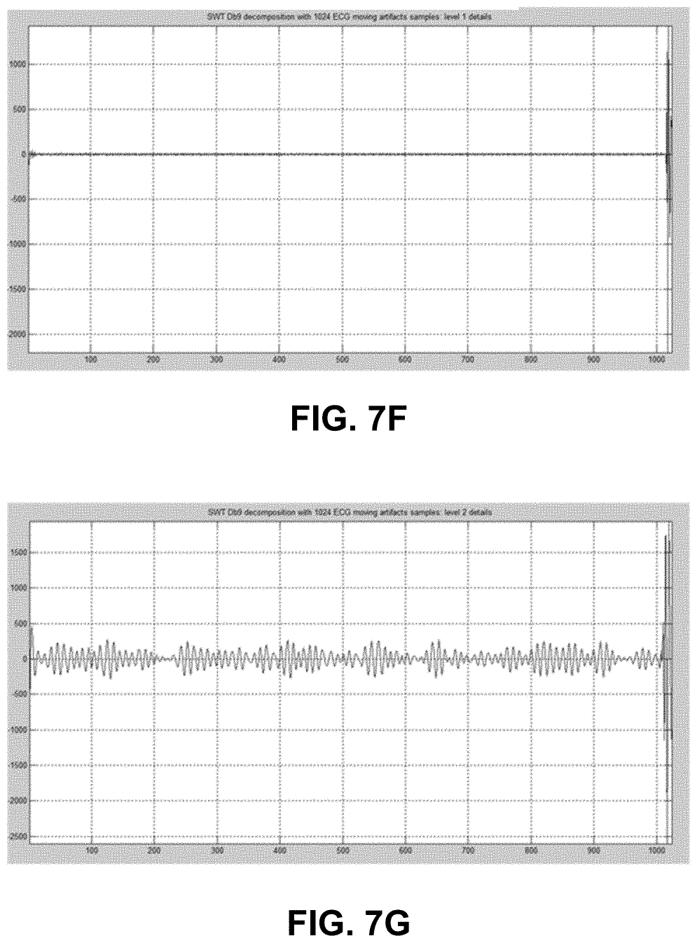

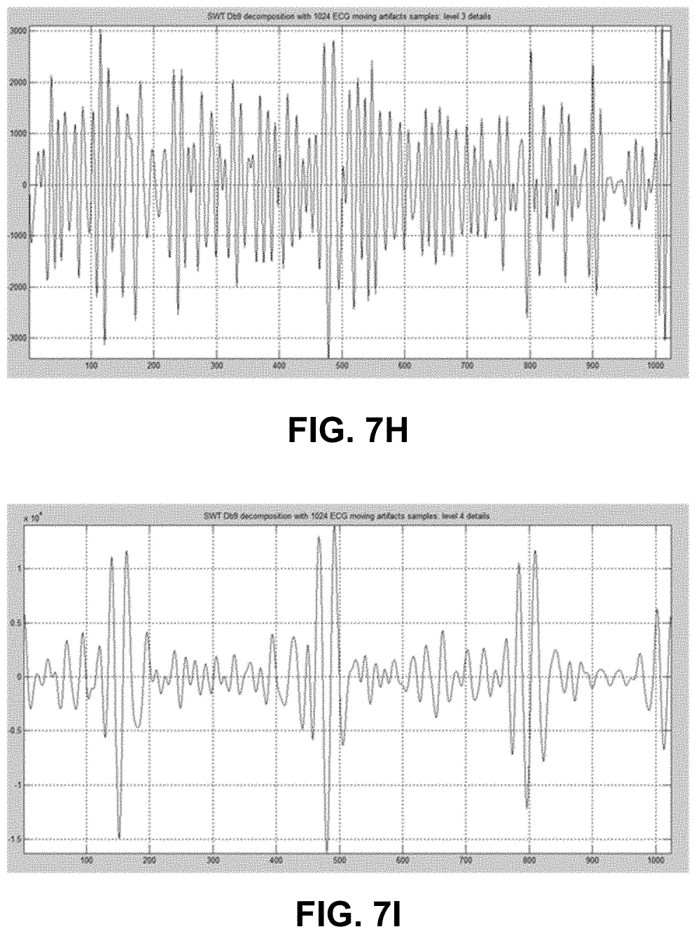

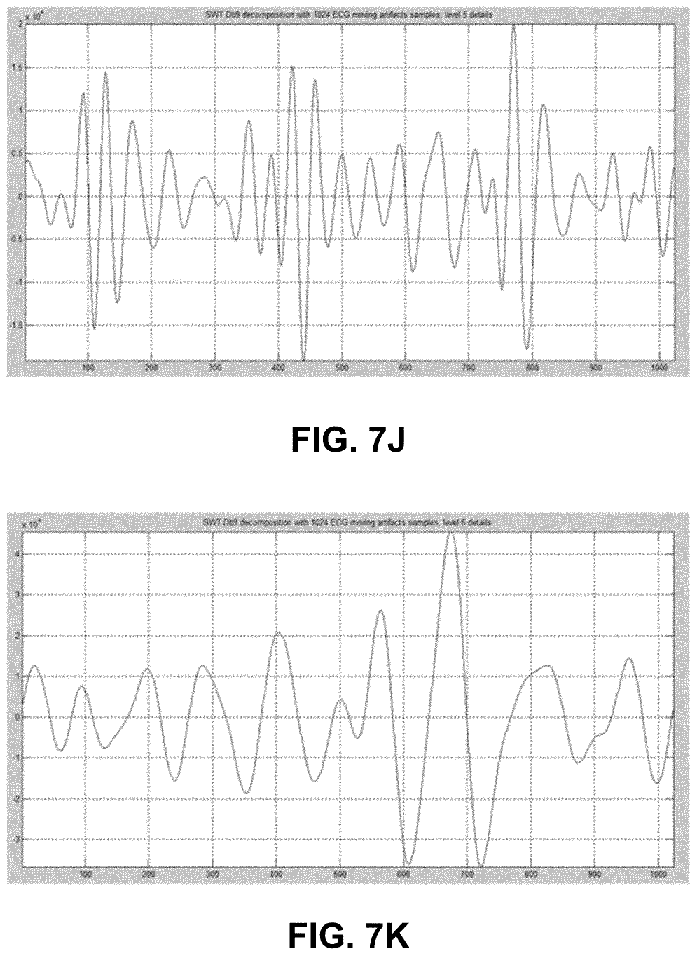

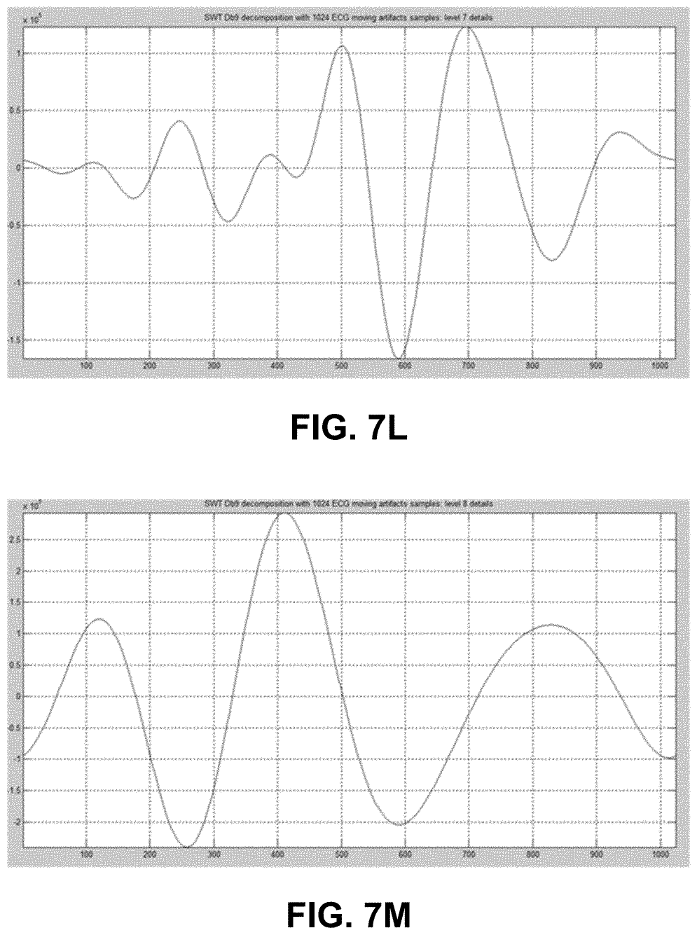

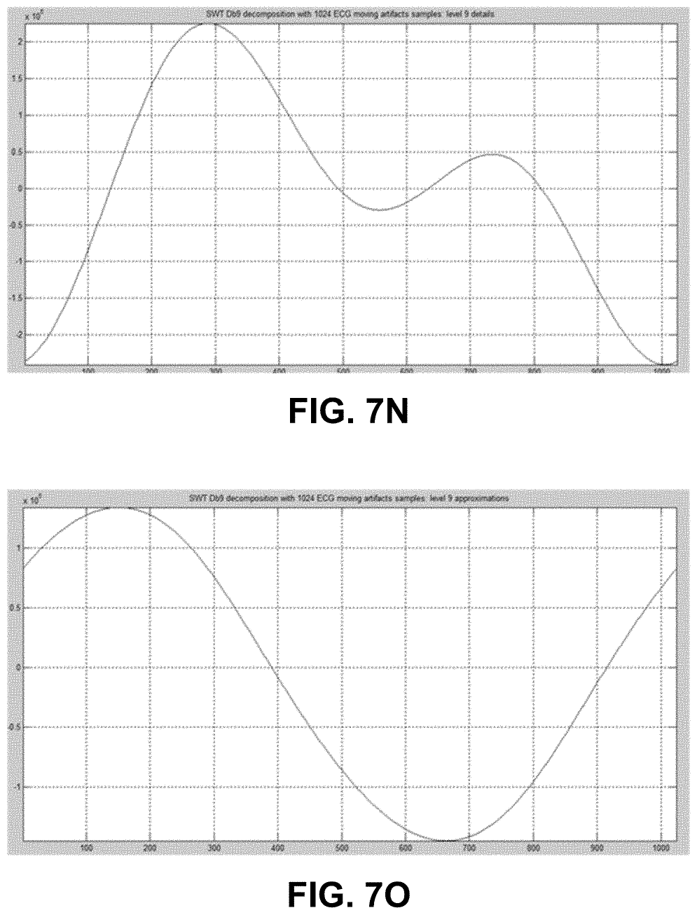

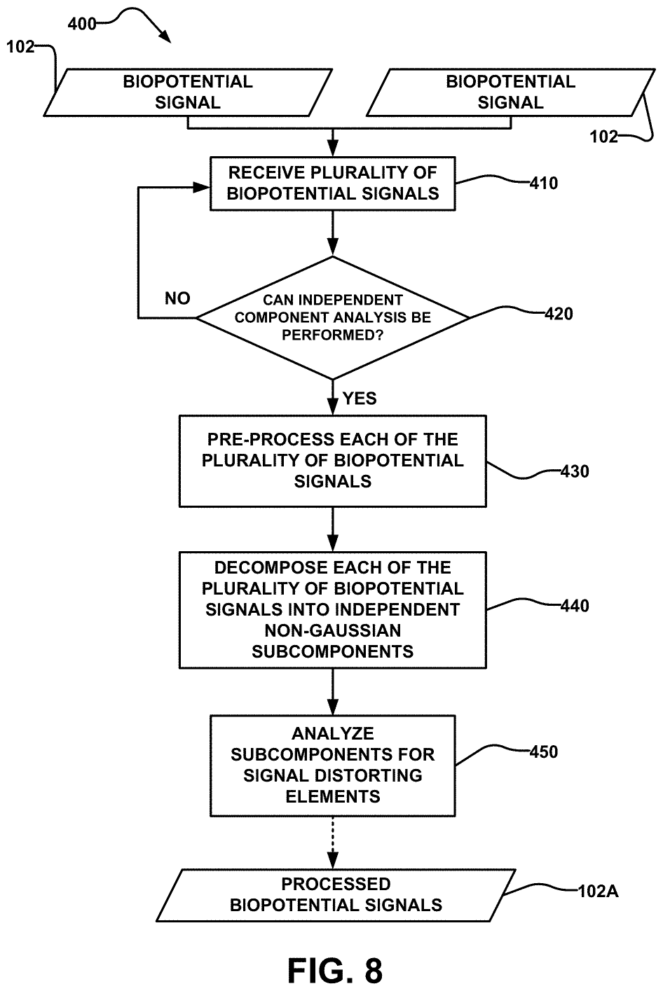

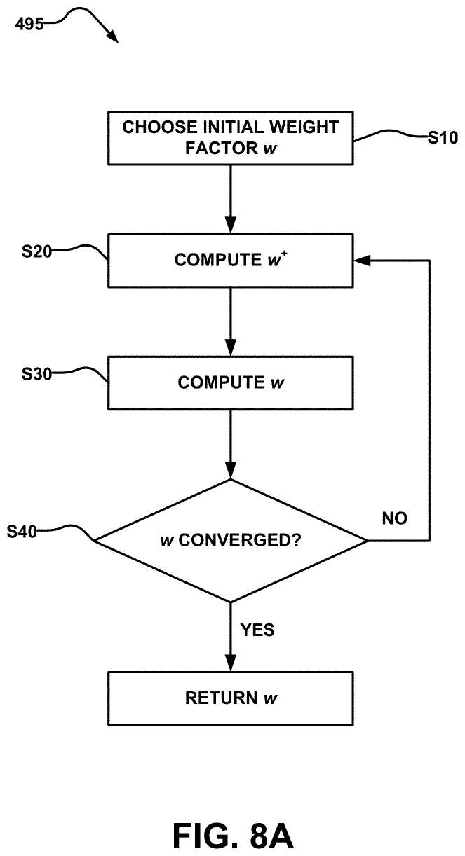

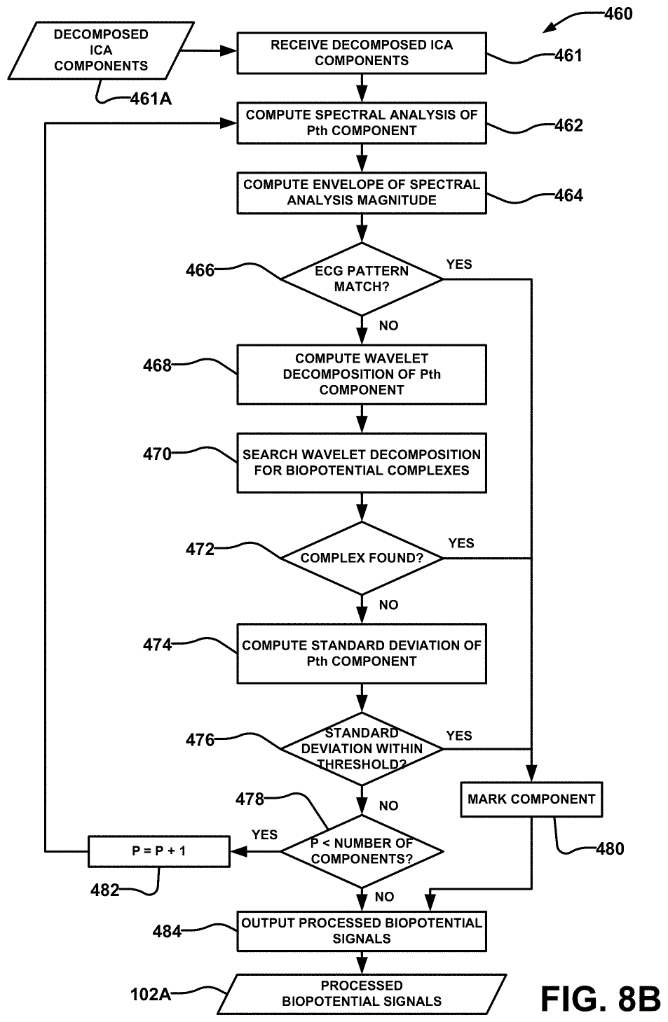

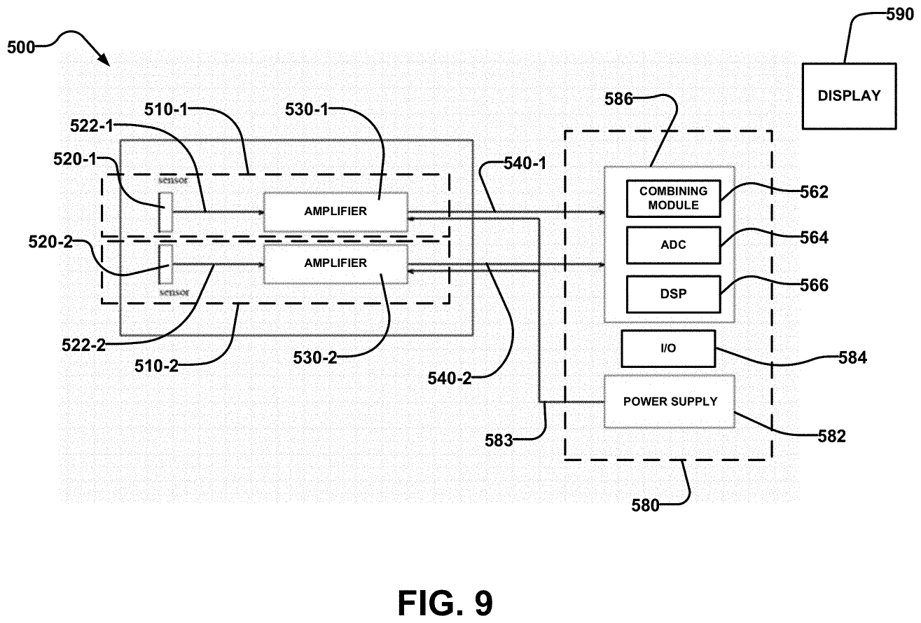

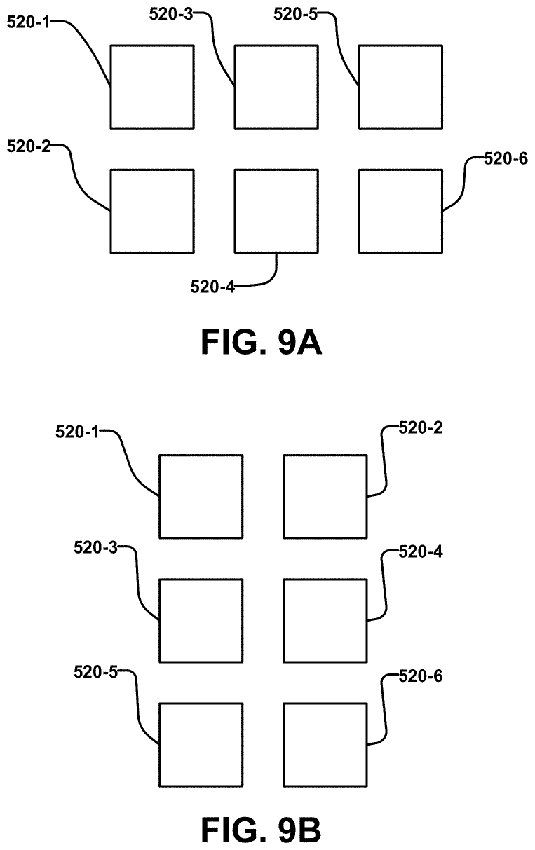

Referring back to FIG. 4, block 160 may additionally or alternatively digitally process biopotential signal 102 using a wavelet method. Such wavelet method may decompose biopotential signal 102 using a wavelet transform, perform thresholding to identify one or more signal distorting elements, suppress the identified signal distorting elements, and reconstruct biopotential signal 102 using an inverse wavelet transform. Such wavelet method may, in addition or alternatively, decompose biopotential signal 102 using a wavelet transform and analyze the decomposed signals directly to determine physiological parameters (e.g. heart rate) corresponding to biopotential signal 102 (e.g. ECG signal). In some embodiments, block 160 may comprise performing example wavelet method 300 illustrated in FIG. 7.

Method 300 commences in block 310 which comprises receiving acquired biopotential signal 102 from block 140. In some embodiments, the received biopotential signal 102 is stored in a buffer. Block 320 verifies that the received biopotential signal 102 comprises sufficient data. For example, in some embodiments, it may be desirable for biopotential signal 102 to comprise at least two continuous QRS complexes (e.g. about 2.048 seconds of ECG data collected at 500 Hz to yield 1024 samples, assuming a lowest possible heart rate of 40 bpm). If block 320 determines that received biopotential signal 102 does not comprise sufficient data, block 320 returns method 300 to block 310. Alternatively, if it is determined that biopotential signal 102 comprises sufficient data (e.g. biopotential signal 102 comprises at least two continuous QRS complexes), method 300 proceeds to block 330.

In block 330, an Nth layer of a wavelet transform is performed. Block 330 may perform any suitable wavelet transform (e.g. a Haar transform, a Daubechies transform, a Biorthogonal transform, a Symlets transform, etc.). In particular, biopotential signal 102 may be decomposed into two portions using a pair of low-pass and high-pass filters. The magnitude response of each filter may, for example, be the mirrored version of the other. In some embodiments, the low and high pass filters are Quadrature Mirrored Filters. The low-pass and high-pass filters may output an Approximation (A) signal and a Detail (D) signal respectively.

Given a biopotential signal 102 (x[k]), outputs of low-pass and high-pass filters with respective impulse responses g and h may, for example, be represented as:

$$\begin{matrix}A = {y}_{l [ n ]} = {\sum }_{k = - ∞}^{k = ∞} x [ k ] g [ n - k ] & ( 6 ) \\ D = {y}_{h [ n ]} = {\sum }_{k = - ∞}^{k = ∞} x [ k ] h [ n - k ] . & ( 7 )\end{matrix}$$

Block 340 may verify that method 300 has decomposed biopotential signal 102 using M layers of low-pass and high-pass filters. In some embodiments, block 340 verifies that biopotential signal 102 has been decomposed using 9 layers of filters (e.g. M=9, but other numbers of layers could be used). If block 340 determines that N is less than M (i.e. the wavelet transform comprises further layers), block 340 returns method 300 to block 330. Otherwise, method 300 proceeds to block 350.

In some embodiments, an approximation signal generated by a first layer of filters will be input into a second layer of filters. As shown in FIG. 7A, an approximation signal 380-1 is input into a second layer of low-pass and high-pass filters. Approximation signal 380-2 is input into a third layer of low-pass and high-pass filters. This process may continue until block 340 determines that a required number of layers has been satisfied.

In some embodiments, a wavelet transform (e.g. a discrete wavelet transform) may result in a time shift in processed biopotential signal 102A as a result of the wavelet transform down sampling biopotential signal 102 as shown in example FIG. 7B. To avoid introducing such time shift into processed biopotential signal 102A, a Stationary Wavelet Transform may, for example, be performed. In such embodiments, zeros may be inserted into filter coefficients at each Nth level of the wavelet transform resulting in a shift-invariant wavelet transform (i.e. the filters at each level are up-sampled versions of the previous). In some embodiments, filter coefficients may be up-sampled by a factor of 2 as shown in example FIG. 7C.

Returning to FIG. 7, in block 350, one or a plurality of wavelet coefficients and/or wavelet levels are analyzed by suitable digital signal processing techniques.

In some embodiments, block 350 analyzes wavelet coefficients of specific levels of the wavelet transform of biopotential signal 102 to extrapolate a corresponding physiological parameter. For example, block 350 may analyze wavelet coefficients of a mid-level (e.g. 4 or 5) wavelet decomposition of an ECG signal to determine the heart rate of an individual.

In some embodiments, block 350 analyzes wavelet coefficients by implementing a dynamic thresholding technique. Dynamic thresholding may involve first identifying a global maximum value of an “input” corresponding to a specific level of a wavelet decomposition of biological signal 102 within a specific timeframe (e.g. 2 seconds) and calculating a threshold level based on the global maximum value. For example, the threshold level th may be calculated as:

th=C*max (8)

where C is a multiplier and max is a global maximum value of the input. C typically has a value in the range of 0.5 to 0.8. In a currently preferred embodiment, C has a value of 0.6. In some embodiments, C is fine-tuned by experiment and/or adjustable in real-time.

Dynamic thresholding may involve determining a Median Absolute Deviation (“MAD”) of the input (in time domain) and calculating the threshold based on the MAD:

th=K*MAD

where K is a multiplier and MAD is the Median Absolute Deviation of the input. K typically has a value in the range of 1.4 to 1.5. In a currently preferred embodiment, K has a value of 1.4826. In some embodiments, K is fine-tuned by experiment and/or adjustable in real-time.

Dynamic thresholding may optionally involve detecting a polarity of the input. Dynamic thresholding may optionally involve inverting the input before identifying the global maximum value. Dynamic thresholding may optionally involve removing (i.e. zeroing) the negative portions (after inverting if inverting is performed) of the input. Zeroing the negative portions of the input advantageously eliminates the possibility of mistaking a positive slope in the negative portions of the input with a positive slope in the positive portions of the input (which may, for example, correspond to the R-wave of an ECG signal).

After calculating threshold level th, dynamic thresholding may involve detecting a positive slope that meets the following requirements:

input[tᵢ]−input[tᵢ₋₁]>S (9.1)

input[tᵢ]>th (9.2)

where tᵢ is a discrete time, input[tᵢ] is an input value at the discrete time, S is a threshold slope value, and th is a threshold level defined in Eq. 8 above. In some embodiments, S is fine-tuned by experiment and/or adjustable in real-time.

After detecting a positive slope that meets the requirements set forth in Eqs. 9.1 and 9.2 above, dynamic thresholding may involve identifying a local maximum value corresponding to the detected positive slope. The local maximum value may be identified by sampling input values for a predetermined period of time (e.g. 40 ms) to locate an input value that meets the following requirements:

input[tᵢ]>input[tᵢ₋₁] (10.1)

input[tᵢ]>input[tᵢ₊₁] (10.2).

In some embodiments, the local maximum values may be processed to determine a physiological parameter in block 190 (see FIG. 4). For example, where biological signal 102 is an ECG signal, the local maximum value may correspond to an R-wave of the ECG signal such that a heart rate may be calculated based on the time duration between two adjacent local maximum values (which correspond to the time duration between two adjacent R-waves).

In some embodiments, block 350 analyzes the ratio of wavelet coefficients of various levels (e.g. level 5 to level 1) to determine the quality of biopotential 102 which may be in the presence of noise and/or artifact. The ratio can be used, for example, to select the best sensor combination (e.g. sensors with lowest noise/artifact, largest signal, etc.) in multi-sensor configurations. The ratio can also be used, for example, to detect a bad quality biopotential 102. Block 350 can optionally reject biopotential 102 with bad quality by, for example, outputting a flat (i.e. all data points set to zero) processed biopotential 102A. Similar ratios can also be applied to EMD as described elsewhere in this application.

In some embodiments, block 350 analyzes the ratios of wavelet coefficients of various combinations of levels (e.g. level 5 to level 1, level 1 to level 4, etc.) to provide a broader view of the quality of biopotential 102 at various frequency ranges.

In some embodiments, block 350 analyzes wavelet coefficients to identify wavelet levels comprising one or more signal distorting elements. Such wavelet levels can be, for example, identified by matching one or more wavelet levels to wavelet levels known to include one or more signal distorting elements. For example, a calibration biopotential signal with known signal distorting elements may be decomposed using one or more wavelet transforms described herein to generate data of wavelet level values corresponding to one or more signal distorting elements. Upon a wavelet level being identified as comprising values corresponding to one or more signal distorting elements, method 300 may optionally proceed to block 360 to apply a thresholding scheme to remove values corresponding to one or more identified signal distorting elements. In some embodiments, block 360 removes all wavelet level values below a threshold value.

Optimum threshold values to be used by block 360 may, for example, be obtained by minimizing an error between the detail (D) coefficients of an original calibrating signal without any artifacts (clean part of the calibrating signal) and the “D” coefficients of the calibration signal with a signal distorting element (e.g. an artifact).

Upon block 360 removing values corresponding to one or more signal distorting elements present in the decomposed wavelet layers, method 300 may optionally proceed to block 370. In block 370, decomposed biopotential signal 102 is reconstructed into a processed biopotential signal 102A using an inverse wavelet transform as shown, for example, in example FIG. 7D. In embodiments where no up-sampling is required (e.g. in embodiments performing a Stationary Wavelet Transform), filter coefficients corresponding to each filter used to perform the inverse wavelet transform may be flipped left to right compared to the filter coefficients used to perform the wavelet transform.

FIG. 7E illustrates an example processed biopotential signal 102A (an ECG signal in this example) generated by processing acquired biopotential signal 102 using a wavelet method as described elsewhere herein. R-Wave 394 of processed biopotential signal 102A corresponds to R-Wave 390 of acquired biopotential signal 102. FIGS. 7F to 7N respectively illustrate Detail signal outputs of each layer corresponding to a nine-layer Wavelet decomposition used to decompose acquired biopotential signal 102 (i.e. level 1 to level 9 respectively). FIG. 70 illustrates an approximation signal output corresponding to the ninth layer of the Wavelet decomposition.

Returning to FIG. 4, in embodiments in which a plurality of biopotential signals 102 is acquired in block 120, block 160 may proceed to suppress one or more signal distorting elements from one or more of the plurality of biopotential signals by performing a method of Blind Source Separation (BSS). The method of BSS may comprise a statistical and/or computational technique that may, for example, decompose a multivariate signal into a plurality of independent non-Gaussian components such as, for example, a method of Independent Component Analysis (ICA).

An example method of ICA may take several input signals (each signal comprising a plurality of sources) and may extract each of the plurality of sources from each signal. In embodiments where the plurality of biopotential signals comprises, for example, ECG signals generated using a plurality of electrodes placed at different locations on a subject's body, each biopotential signal may comprise a plurality of sources such as, for example, ECG data, noise (e.g. 60 Hz electromagnetic interference) and/or artifacts (e.g. motion artifacts, etc.). Each biopotential signal may comprise the same and/or different sources compared to the other biopotential signals in the plurality of biopotential signals. A method of ICA may, for example, extract ECG data while suppressing other sources (e.g. noise sources, artifact sources, etc.).

In some embodiments, ICA may be performed according to example method 400 shown in FIG. 8.

Method 400 commences in block 410 which comprises receiving a plurality of biopotential signals 102 from block 140. The plurality of biopotential signals (e.g. x[k]) may, for example, be represented as:

$$\begin{matrix}{x}_{1} [ k ] = {a}_{11} {s}_{1} + {a}_{12} {s}_{2} + … + {a}_{1 j} {s}_{j} & ( 11 ) \\ {x}_{2} [ k ] = {a}_{21} {s}_{1} + {a}_{22} {s}_{2} + … + {a}_{2 j} {s}_{j} & ( 12 ) \\ ⋮ & \\ {x}_{i} [ k ] = {a}_{i 1} {s}_{1} + {a}_{i 2} {s}_{2} + … + {a}_{i j} {s}_{j} & ( 13 )\end{matrix}$$

where xᵢ[k] represents a biopotential signal 102 in the plurality of biopotential signals, aᵢⱼ represents weighting parameters (e.g. may depend on placement of electrodes used to generate the plurality of biopotential signals) and sₛ represents each signal source that forms xᵢ[k] (e.g. biopotential data, noise, artifacts, etc.). In some embodiments, xᵢ[k] additionally comprises one or more phase delays between signal sources sₛ. However, in embodiments where the plurality of acquired biopotential signals comprises low frequency signals and distances between tissue surfaces of a subject and sensing surfaces of electrodes used to acquire the plurality of biopotential signals are short, it may be assumed that any phase delays between signal sources sⱼ are negligible.

A plurality of biopotential signals comprising n linear mixtures x₁, x₂, . . . , xₙ of independent components:

xᵢ=aᵢ₁s+aᵢ₂s₂+ . . . aᵢₙsₙ (14)

may, for example, be represented as:

x=As (15)

where x represents a random vector comprising elements that are the mixtures xᵢ of independent components, s represents a random vector comprising elements sₛ and A represents a matrix comprising weighting parameters aᵢⱼ.

When performing an ICA method described herein, it may be assumed that each mixture xᵢ and each independent component sⱼ is a random variable. In preferred embodiments, each mixture xᵢ and each independent component sⱼ have zero mean. Alternatively, each mixture xᵢ and each independent component sⱼ may be centered as described elsewhere herein for each mixture xᵢ and each independent component sⱼ to have zero mean.

Matrix A may, for example, be estimated using observed vector x. Assuming elements sⱼ are statistically independent, have non-gaussian distributions and matrix A is a square matrix, an inverse matrix W can be computed from estimated matrix A. In such embodiments, independent components s may be determined as follows:

s=Wx (16).

Returning to method 400 (FIG. 8), upon at least two biopotential signals 102 being received, method 400 proceeds to block 420 which comprises verifying that ICA method 400 may be performed using the received plurality of biopotential signals 102. For example, for proper application of ICA method 400, it may be desirable that the plurality of biopotential signals 102 was acquired using different sensors (i.e. at least two different electrode combinations). In addition, it may be desirable that each component of each biopotential signal 102 of the plurality of biopotential signals is non-Gaussian and is independent of any other component. If one or more of such conditions for performing example ICA method 400 on the received plurality of biopotential signals is not satisfied, method 400 may return to block 410. Conversely, if all of these conditions for performing ICA method 400 are satisfied, method 400 proceeds to block 430.

In block 430, each biopotential signal 102 of the plurality of biopotential signals is pre-processed. Advantageously, pre-processing each biopotential signal 102 may, for example, increase computational efficiency of method 400.

In some embodiments, block 430 comprises centering vector x. As described elsewhere herein, centering vector x may simplify ICA estimation (e.g. increase computational efficiency of method 400). Vector x may, for example, be centered by making vector x a zero-mean variable. Vector x may, for example, be converted into a zero-mean variable by subtracting a mean vector m=E{x}, from vector x. In such embodiments, zero-centering vector x necessarily implies that s is also a zero-mean variable. The mean of s may, for example, be given by A⁻¹m, where m is the mean vector that was subtracted in the centering of vector x. Centered versions of vectors x and s, x′ and s′ respectively may, for example, be represented as:

x′=x−E{x} (17)=

s′=s−A⁻¹E{x} (18).

Alternatively, or in addition, block 430 may whiten vector x. Advantageously, whitening generates a mixing matrix that is orthogonal. Having a mixing matrix that is orthogonal may, for example, be advantageous as the number of parameters to be estimated is reduced by half (i.e. an orthogonal matrix comprises n(n−1)/2 free parameters). Whitening vector x, for example, may comprise transforming vector x linearly to obtain a new vector x comprising uncorrelated components with unity variances (i.e. variances equal to one). In such embodiments, a covariance matrix of x is equivalent to the identity matrix I and may, for example, be represented as follows:

E{\tilde{x}\tilde{x}^{T}}=I (19).

New vector \tilde{x} may, for example be represented as:

\tilde{x}=ED^{-1/2}E^{T}x (20)

where the columns of E and the diagonal of D are the eigenvectors and eigenvalues of E{xx^{T}}, which may, for example, be represented as:

E{xx^{T}}=EDE^{T} (21).

Alternatively, or in addition, block 430 may filter the plurality of biopotential signals using, for example, low-pass filtering, high-pass filtering, band-pass filtering, band-stop filtering and/or the like. Such filtering may, for example, remove frequency bands which fall outside of a range of frequencies which may comprise desired biopotential data to be extracted from the plurality of biopotential signals.

Upon each of the plurality of biopotential signals being pre-processed in block 430, method 400 proceeds to block 440. In block 440, each of the plurality of biopotential signals is decomposed into its independent non-Gaussian subcomponents (i.e. sources). Each of the plurality of biopotential signals may be decomposed successively (i.e. one after the other) or simultaneously (i.e. at the same time). In some embodiments, one subcomponent of each of the plurality of biopotential signals is decomposed prior to block 440 proceeding to decompose a subsequent subcomponent of each of the plurality of bipotential signals 102.

In some embodiments, block 440 decomposes each of the plurality of biopotential signals into their independent non-Gaussian subcomponents by maximizing a contrast function (i.e. a function measuring independence of random variables, such as, for example, a measure of non-Gaussianity or any other measure of independence).

In some embodiments block 440 may measure non-Gaussianity by performing a method of kurtosis. For a Gaussian random variable, kurtosis is zero. kurtosis may, for example, be represented as:

kurt(y)=E[y⁴]−3(E[y²])² (22).

If it is assumed that y is of unit variance (i.e. a variance value equal to one), the right side simplifies to E[y⁴]−3. For a gaussian y, the fourth moment equals 3(E[y²])², and thus the kurtosis of y becomes zero. Advantageously, measuring non-Gaussianity using kurtosis may increase computationally efficiency of method 400. In some embodiments, however, kurtosis may be sensitive to outliers.

In some embodiments, block 440 measures non-Gaussianity by negentropy (i.e. based on an information-theoretic quantity of entropy and/or differential entropy). If all random variables are of equal variance, then a Gaussian variable will have the largest entropy. Negentropy is zero if and only if y has a gaussian distribution (otherwise, negentropy is always non-negative).

The entropy of a variable can be thought of as a measure of its randomness. For a discrete random variable Y, entropy H may, for example, be represented as:

H(Y)=−Σᵢ₌₁ⁿP(yᵢ)log_{b}P(yᵢ) (23).

A differential entropy of a random (continuous-valued) vector y with density function ƒ (y) may, for example, be represented as:

H(y)=−∫ƒ(y)log ƒ(y)d_{y} (24).

Negentropy J may, for example, be represented as:

J(y)=H(y_{gauss})−H(y) (25).

where y_{gauss} is a Gaussian random variable of the same covariance matrix as y.

Estimating negentropy may be computationally intensive as it requires, for example, an estimate of a probability density function (PDF). In some embodiments, block 440 may by simplified (i.e. made more computational efficient) by using an approximation of negentropy to measure non-Gaussianity. Such approximation may comprise an approximation proposed by Hyvarinen which may, for example, be represented as:

J(y)∝[E{G(y)}−E{G(v)}]² (26)

where v is a Gaussian variable of zero mean and unit variance, and G is any suitable non-quadratic function. Examples of G that have been shown to work well include:

$$\begin{matrix}{G}_{1} ( u ) = \frac{1}{{a}_{1}} log cosh ( {a}_{1} u ) ; where 1 \leq {a}_{1} \leq 2 & ( 27 ) \\ {G}_{2} ( u ) = - exp ( - {u}^{2} / 2 ) . & ( 28 )\end{matrix}$$

Block 440 may, for example, maximize the contrast function by performing, for example, FastICA. In some embodiments, a single-unit FastICA is performed (i.e. FastICA for one computational neuron comprising a weight vector w that is updated by the neuron based on a learning rule). In some embodiments, a multi-unit FastICA is performed (i.e. FastICA for embodiments comprising a plurality of computational neurons).

Assuming block 430 has centered and whitened each of the plurality of biopotential signals 102, a single-unit FastICA learning rule (i.e. a rule used to train the computational neuron) may find a vector w such that the projection w^{T}x maximizes non-gaussianity. Non-gaussianity may, for example, be measured by the approximation of negentropy J(w^{T}x):

J(w^{T}x)∝(E[G(w^{T}x)]−E[G(v)])² (29).

The maxima of J(w^{T}x) occurs at, for example, certain optima of E{G(w^{T}x)}. The second part of the estimate (e.g. E[G(v)]) is independent of w. According to Kuhn-Tucker conditions, the optima of E{G(w^{T}x)} with the constraint E{(w^{T}x)²}=∥w∥²=1 occurs at the points where, for example:

F(w)=E[xg(w^{T}x)]−βw=0 (30)

where g(u)=dG(u)/du. E{(w^{T}x)²}=∥w∥² is constrained to 1 as variance of w^{T}x must be equal to unity (as the data (e.g. the plurality of biopotential signals) was whitened, the norm of w must be equal to 1). To solve equation (27) and find w, the problem may, for example, be approximated as a Newton's iteration. In such embodiments, to find a zero of a function ƒ(x), the following iteration is applied:

xₙ=xₙ−ƒ(xₙ)/ƒ′(xₙ) (31).

The Jacobian of F(w) becomes:

JF(X)=E{xx^{T}g′(w^{T}x)}−βI (32).

Block 440 may, for example, approximate the first term of the above expression by noting that the data is sphered, and therefore simplify the inversion of the following matrix:

E[xx^{T}g′(w^{T}x)]≈E[xx^{T}]E[g′(w^{T}x)]=E[g′(w^{T}x)]I (33).

The term E[g′(w^{T}x)] is a scalar, so the Jacobian is diagonal which simplifies the inversion, and therefore, the approximate Newton's iteration may become:

w⁺=w−(E[xg(w^{T}x)]−βw)/(E[g′(w^{T}x)]−β) (34).

By multiplying both sides of Equation (31) by β−E[g′(w^{T}x)], the FastICA iteration may comprise (see example FastICA iteration 495 shown in FIG. 8A):
1) choosing an initial weight vector w (e.g. block S10),
2) computing w⁺, where w⁺ may, for example be represented as: w⁺=E{xg(w^{T} x)}−E{g′ (w^{T} x)}w (e.g. block S20);
3) computing w, where w may, for example be represented as: w=w⁺/∥w⁺∥ (e.g. block S30); and
4) if the FastICA has not converged, repeat the above from step 2 onwards (e.g. block S40).

In some embodiments, block 440 may estimate a plurality of ICA components by performing the example single-unit FastICA method described elsewhere herein using several units comprising weights w₁, w₂, . . . wₙ (i.e. a “multi-unit FastICA”). Such embodiments may, for example, result in outputs w₁^{T}, w₂^{T}, . . . , wₙ^{T}. Outputs w₁^{T}, w₂^{T}, . . . , wₙ^{T} may be de-correlated at each iteration to prevent several of these vectors from converging to the same solution. Such multi-unit FastICA may, for example, be based on a Gram-Schmidt deflation scheme as follows:
1) estimate each independent component one by one;
2) with p estimated components w₁, w₂, . . . , wₚ, a single-unit ICA iteration is run for wₚ₊₁;
3) after each iteration, subtract the projection of wₚ₊₁ on the previous vectors wⱼ; and
4) renormalize wₚ₊₁.

Optionally, the aforementioned Gram-Schmidt deflation scheme may, for example, be formulaically represented as:

1) wₚ₊₁=wₚ₊₁−Σⱼ₌₁^{p}wₚ₊₁^{T}wⱼwⱼ; and

2) wₚ₊₁=wₚ₊₁/\sqrt{wₚ₊₁^{T}wₚ₊₁}.

In some embodiments, such multi-unit FastICA may comprise computing all components simultaneously (i.e. no weighting vectors are privileged over others). Such embodiments may be advantageous in applications where, for example, a symmetric decorrelation is needed. Such symmetric decorrelation may be accomplished by matrix square roots as follows:

W=(WW^{T})^{-1/2}W (35).

where W is the matrix (w₁, w₂, . . . , wₙ)^{T} of the vectors, and an inverse square root is obtained using eigenvalue decomposition of WW^{T}=FDF^{T} as (WW^{T})^{-1/2}=FD^{-1/2}F^{T}. A simpler alternative may, for example, be to perform the following iteration algorithm by Hyvarinen:

1) W=W/\sqrt{∥WW^{T}∥}; and

2) W=3/2W−½WW^{T}W (repeat until convergence).

In some embodiments, block 440 decomposes each of the plurality of biopotential signals 102 into its independent non-Gaussian subcomponents by minimization of mutual information. In such embodiments, the mutual information I between m scalar random variables yᵢ, i=1 . . . m may, for example, be defined based on a concept of differential entropy as follows:

I(y₁,y₁₂, . . . ,y₁ₘ)=Σᵢ₌₁^{m}H(yᵢ)−H(y) (36).

where H( ) denotes entropy, and y is a random vector with density ƒ(y) such that:

H(Y)=−∫ƒ(y)log ƒ(y)d_{y} (37).

Mutual information is a natural way to measure dependence between random variables. Mutual information will be zero if and only if variables are statistically independent. Otherwise, mutual information will be non-negative. Block 440 may, for example, define an ICA of random vector x as an invertible transformation (i.e. s=Wx) and proceed to determine matrix W such that the mutual information of the transformed components sᵢ is minimized.

Minimization of mutual information may, for example, be roughly equivalent to finding directions where negentropy is maximized, or equivalent to maximizing the sum of non-Gaussianities of the estimates (that are constrained to be uncorrelated).

Once each of the plurality of biopotential signals is fully decomposed, method 400 proceeds to block 450. In block 450, the decomposed individual subcomponents of each of the plurality of biopotential signals corresponding to artifacts and/or noise are identified. Such components may, for example, be identified based on a frequency spectrum, amplitude thresholds, recognized patterns and/or the like. Components identified as corresponding to artifacts and/or noise are suppressed in block 450 during reconstruction of each of the plurality of biopotential signals. In some embodiments, each of the plurality of biopotential signals may be reconstructed in real time.

In some embodiments, block 450 identifies ICA components in real time using example method 460 shown in FIG. 8B. Method 460 commences in block 461 which receives a plurality of decomposed ICA components 461A. Method 460 then proceeds to block 462 which comprises performing a spectral analysis (e.g. a Fast Fourier Transform (FFT)) of a first decomposed ICA component (i.e. source). In block 464, an envelope of a magnitude of the computed spectral analysis (e.g. a computed FFT) is determined. In block 466, the computed spectral analysis and envelope are used to match the first decomposed ICA component to a biopotential pattern (e.g. an ECG pattern). If a match is made, method 460 proceeds to block 480 which comprises marking the identified ICA component. Otherwise, method 460 proceeds to block 468.

In block 468, a wavelet decomposition of the first decomposed ICA component is performed. For example, such wavelet decomposition may comprise a 6-9 level stationary wavelet transform in some embodiments, although different numbers of levels may be used. Method 460 then proceeds to block 470 which comprises searching the results of the wavelet decomposition for biopotential complexes. For example, a QRS complex of ECG data, if present, typically appears in levels 3-4 of the wavelet decomposition. If the length of the data is known, a time-domain analysis of the wavelet decomposition levels may be performed to identify, for example, an R-Wave of ECG data. In block 472, method 460 determines if a biopotential complex has been found. If so, method 460 proceeds to block 480 described elsewhere herein. Otherwise method 460 proceeds to block 474.

In block 474, a standard deviation of the first decomposed ICA component is determined. When combined with thresholding, for example, computed standard deviation values may be used to differentiate ICA components corresponding to biopotential data from ICA components corresponding to non-biopotential data (e.g. noise, artifacts, etc.). For example, motion artifacts in a non-contact ECG system are typically at least an order of magnitude larger than a peak-to-peak average of ECG signal data. In block 476, method 460 determines whether the computed standard deviation corresponds to biopotential data. If so, method 460 proceeds to block 480 described elsewhere herein. Otherwise, method 460 proceeds to block 478.

Block 478 determines if further decomposed ICA components are to be analyzed. If so, method 460 proceeds to block 482 which comprises selecting the next decomposed ICA component to be analyzed. Method 460 then returns to block 462. If no further ICA components are to be analyzed, method 460 proceeds to block 484 which comprises outputting the decomposed ICA components marked in block 480. Components not marked as corresponding to biopotential data (i.e. identified as corresponding to artifacts and/or noise) are suppressed in block 484 during reconstruction of each of the plurality of biopotential signals 102 by for example, removing such components from the reconstruction. Block 484 outputs each of the reconstructed plurality of biopotential signals as a processed biopotential signal 102A.

Method 460 may optionally comprise performing all of, some of or only one of the described spectral analysis, wavelet decomposition and standard deviation analysis. In some embodiments, method 460 may only perform the wavelet decomposition to, for example, identify one or more QRS complexes and/or R-waves present in ECG data. In some embodiments, method 460 may, for example, perform the described spectral analysis and standard deviation analysis but not the described wavelet decomposition.

Returning to method 100 shown in FIG. 4, processed biopotential signal 102A is optionally output in block 180. In some embodiments, processed biopotential signal 102 may be displayed to a user using a display. Alternatively, or in addition, processed biopotential signal 102 may be printed, communicated to a user using a suitable network interface, stored locally and/or remotely, communicated to a processor for further processing using a suitable network interface, or the like.

In block 190, one or more physiological parameters (e.g. heart rate) are calculated based on processed biopotential signal 102A. In some embodiments, processed biopotential signal 102A comprises decomposed components of biopotential signal 102. In some embodiments, processed biopotential signal 102A comprises a signal reconstructed from the decomposed components of biopotential signal 102 after suppressing one or more signal distorting elements from the decomposed components.

In some embodiments, block 190 calculates the heart rate of an individual based on a processed ECG signal. In some embodiments, block 190 verifies that the calculated heart rate is within a minimum possible heart rate (e.g. 40 bpm) and a maximum possible heart rate (e.g. 240 bpm). Where the calculated heart rate falls outside of the range of possible heart rates bounded by the minimum possible heart rate and the maximum possible heart rate, block 190 may indicate that the calculated heart rate is inaccurate.

In some embodiments, method 100 is performed continuously in real-time. In such embodiments, biopotential signal 102 is optimized and output in real-time while a subject remains coupled to a system for acquiring biopotential signal 102. In some embodiments, method 100 may be performed in a plurality of distinct stages. For example, method 100 may acquire biopotential signal 102 (e.g. block 120 described elsewhere herein) during a first stage while a subject is coupled to a system for acquiring biopotential signal 102. Method 100 may then proceed to optimize and output biopotential signal 102 during a second stage when the subject is no longer coupled to the system for acquiring biopotential signal 102.

Signal distorting elements may, for example, extend from a few hundred samples (e.g. several hundred milliseconds in a 500 sps ECG system) to about 3000 samples (e.g. 6 seconds of data at 500 sps). In some embodiments, any processing method described herein can suppress such signal distorting elements of varying lengths. In some embodiments, any processing method described herein comprises a buffer large enough to accommodate at least 3000 samples. In some embodiments, buffer sizes may be dynamically adjusted to accommodate varying lengths of signal distorting elements that may be present in a biopotential signal 102. Dynamically varying buffer size may, for example, improve computation efficiency.

FIG. 9 is a schematic illustration of an example biopotential measurement system 500 according to a particular embodiment. In some embodiments, biopotential measurement system 500 comprises a plurality (e.g. a pair in the illustrated embodiment) of electrode systems 510-1, 510-2 which may be used, for example, to acquire a biopotential signal 102 (e.g. a single-lead ECG). Electrode system 510-1 comprises electrode 520-1 and amplifier circuit 530-1. Electrode system 510-2 comprises electrode 520-2 and amplifier circuit 530-2. Electrodes 520-1, 520-2 (each an electrode 520) may be contact or contactless electrodes. Amplifier circuits 530-1, 530-2 may, for example, condition (e.g. amplify, filter, etc.) signals 522-1 and/or 522-2 generated by electrodes 520-1, 520-2 respectively. Outputs of amplifier circuits 530-1, 530-2 are communicatively coupled to a base unit 580 for amplified signals 540-1, 540-2 to be transmitted to base unit 580. In some embodiments, amplified signals 540-1, 540-2 are transmitted to base unit 580 using a suitable wireless interface. In some embodiments, amplified signals 540-1, 540-2 are transmitted to base unit 580 using a suitable wired interface.

Base unit 580 comprises power supply 582, I/O module 584 and processing module 586. Power supply 582, for example, generates power signal 583 used to power one or more electrode systems 510. I/O module 584 may comprise one or more output devices for outputting data (e.g. a processed biopotential signal 102A) to a user such as, for example, one or more displays (e.g. display 590), a printer or the like. I/O module 584 may also comprise one or more input interfaces for receiving data (e.g. a pre-recorded biopotential signal 102, particulars of a subject, etc.) from a user such as, for example, a touch-screen, a keyboard, a mouse, a usb interface or the like. In some embodiments, I/O module 584 comprises a suitable network interface for communicating data (e.g. biopotential signal 102, processed biopotential signal 102A) to and/or from base unit 580 via a suitable network.

In a preferred embodiment, processing module 586 comprises combining module 562, analog to digital converter 564 and digital signal processing module 566. Combining module 562 combines amplified signals 540 (e.g. signals 540-1, 540-2) to generate one or more biopotential signals 102. Analog to digital converter 564 transforms biopotential signal 102 to a digital domain using a resolution required by digital signal processing module 566. Analog to digital converter 564 may comprise any commercially available analog to digital converter (ADC). In preferred embodiments, analog to digital converter 564 comprises a multi-channel synchronous ADC or a plurality of synchronized single-channel ADCs synchronized to sample simultaneously (e.g. a plurality of synchronized one-channel ADCs). In such embodiments, a sampling phase delay (which may skew processing of biopotential signal 102) may be minimized, or completely eliminated. Digital signal processing module 566 may suppresses one or more signal distorting elements from biopotential signal 102 generating a processed biopotential signal 102A using any method described elsewhere herein (e.g. block 160 of method 100).

In some embodiments, one or more of combining module 562, analog to digital converter 564 and digital signal processing module 566 may be independent of base unit 580 (i.e. may form intermediary components between electrode systems 510 and base unit 580).

In another embodiment, biopotential measurement system 500 may comprise three contactless electrode systems 510-1, 510-2, 510-3 (not explicitly shown). A standard 3-lead ECG may be measured, for example, by coupling electrodes 520-1, 520-2 and 520-3 (not explicitly shown) to a subject's right arm (RA), left arm (LA) and left leg (LL) respectively. In another embodiment of biopotential measurement system 500, an EEG may be measured by, for example, further increasing a number of electrode systems 510-1 . . . 510-P used by bio-potential measurement system 500.

In some embodiments where, for example, ICA method 400 is performed, biopotential measurement system 500 may comprise six or more electrodes 520 (contact or contactless) to allow for a standard three-lead configuration. For example, the at least six electrodes 520 may be positioned in an array of 2 rows×3 columns or 3 rows×2 columns as shown respectively in FIGS. 9A and 9B. Using at least six electrodes allows ICA method 400 to, for example, detect and suppress all signal distorting elements. In some such embodiments, biopotential measurement system 500 may comprise a maximum of 16 electrodes 520.

Advantageously, digital signal processing module 566 may be used to suppress one or more signal distorting elements (e.g. artifacts, noise or the like) that may be present in one or more acquired biopotential signals 102. As described elsewhere herein, in some embodiments such signal distorting elements may completely mask desired components of one or more biopotential signals 102 (e.g. may mask a QRS complex of an ECG signal, etc.).

In some embodiments, biopotential measurement system 500 described herein may be implemented in a vehicular setting (e.g. inside a car, truck, bus, plane, boat or the like). Such embodiments may comprise embedding one or more electrodes 520 into components of the vehicle, such as (without limitation): the vehicle seat(s), seat restraints, the steering wheel, the dashboard, the vehicle ceiling, the vehicle floor and/or the like. Embedded electrodes 520 may, for example, be used to determine the state of subject's heart muscle (i.e. ECG measurement) and/or the skeletal or other muscle (i.e. EMG measurement) of the vehicle operator. Such information may be communicated to first responders or suitable authorities in the event of an accident or during normal vehicular operation periods. Such embodiments can also alert a vehicle operator (e.g. using suitable alarms or the like) that the vehicle operator is having a cardiac event (e.g. a heart attack) or similar heart condition. Data from such vehicular ECG systems and/or EMG systems may be recorded—e.g. for forensic analysis, data analytics or the like. In some embodiments, data from such vehicular ECG systems and/or EMG systems may be used to adjust the vehicle seat(s), steering wheel, seat warmer(s), seat vent(s), air conditioning settings, or the like. In some embodiments, different emotional states (e.g. a stressed state, a relaxed state, etc.) detected using such data may trigger different adjustments (e.g. a vehicle seat may be adjusted differently depending on a detected emotional state, air-conditioning settings may be set to different temperatures depending on whether a subject is in a stressed state or a relaxed state, etc.).

In some embodiments, one or more biopotential signals 102 corresponding to, for example, ECG measurements may, for example, be analyzed to determine respiration patterns of a subject. In such embodiments, the respiration information may be used alone or in conjunction with ECG data or other data (e.g. EEG data, EMG data or EOG data) to determine a state of a subject, such as, for example, whether the subject is asleep, drowsy, impaired, is suffering from medical conditions or the like.

In some embodiments, one or more biopotential signals 102 may be analyzed alone or in combination with other signals to determine a medical state of a subject and/or provide analytics related to, for example, drowsiness, unconsciousness, incapacity, brain injury, stroke, arrhythmias, compensated shock, decompensated shock, sepsis, heart attack, sleep apnea, stress, attentiveness, cognition, respirations, internal bleeding, body temperature, personal identification, electrolyte imbalance, or the like.

In some embodiments, one or more biopotential signals 102 may be analyzed alone or in combination with other signals to identify a subject. For example, a biopotential signal 102 may be compared against one or more known signals (ECG signals, EEG signals, EMG signals, EOG signals, etc.), each signal representative of a different subject's identity. In some embodiments, biopotential signal 102 is an ECG signal. In such embodiments, differences in parameters such as resting heart rates, QRS complexes, etc. may, for example, be used to match biopotential signal 102 to (or differentiate biopotential signal 102 from) one or more ECG signals representative of different identities.

In some embodiments, a vehicle embedded system as described elsewhere herein may ascertain the identities of the vehicle operator and/or passenger(s). Upon ascertaining the identities, the vehicle may, for example, automatically adjust the vehicle seat(s), steering wheel, environmental conditions or the like according to each of the identified subject's pre-configured preferences.

In some embodiments, software may be used to interpret one or more biopotential signals 102 to provide detailed information about the state of a subject.

In some embodiments, a biopotential measurement system 500 may be incorporated or embedded into devices such as, for example, cellular phones, tablets, laptop computers, desktop computers, smart watches, activity trackers, animal vests, animal beds, infant hospital beds, infant incubators or the like and/or casing or other protective gear for such devices. In some embodiments, a biopotential measurement system 500 may be incorporated or embedded into, for example, hospital beds, gurneys, wheel-chairs, medical examination tables, household furnishings including household bed frames or the like.

In some embodiments, the systems and methods described herein are not limited to humans and may be used for measurement of electrical activity within animals, such as, for example, pet animals, zoo animals, rescued wild animals, wild animals or the like. Accordingly, unless the context clearly requires otherwise, throughout the description and the claims, “subject” is to be construed as inclusive of both human subjects as well as animal subjects.

In some embodiments, where one or more biopotential signals 102 relates to the operation of cell(s), tissue(s), organ(s) and/or system(s), biopotential measurement system 500 may be configured to use these signals (individually and/or together) to create and display animation on a suitable display (e.g. display 590). The displayed animation may be based on one or more biopotential signals 102 and may, for example, show the operation of the cell(s), tissue(s), organ(s) and/or system(s).

In some embodiments, breathing artifacts present in a biopotential signal 102 may, for example, be enhanced using one or more of the methods described herein (i.e. the methods described herein are used to suppress signal elements other than the breathing artifacts). In such embodiments, the breathing artifacts may be used to determine physiological data such as a respiratory rate of a subject.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof; elements which are integrally formed may be considered to be connected or coupled;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list; and
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a computer system for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Embodiments of the invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g. EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e. that is functionally equivalent), including components which are not structurally equivalent to the disclosed structure which performs the function in the illustrated exemplary embodiments of the invention.

Where a record, field, entry, and/or other element of a database is referred to above, unless otherwise indicated, such reference should be interpreted as including a plurality of records, fields, entries, and/or other elements, as appropriate. Such reference should also be interpreted as including a portion of one or more records, fields, entries, and/or other elements, as appropriate. For example, a plurality of “physical” records in a database (i.e. records encoded in the database's structure) may be regarded as one “logical” record for the purpose of the description above and the claims below, even if the plurality of physical records includes information which is excluded from the logical record.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A method for suppressing one or more signal distorting elements from a biopotential signal indicative of a biopotential at a location on a body of a subject, the method comprising:
acquiring the biopotential signal from the body of the subject using a plurality of electrodes; and
digitally processing the biopotential signal, the digital processing comprising:
decomposing the biopotential signal into a plurality of subsignals;
identifying the one or more signal distorting elements in the plurality of subsignals; and
reconstructing the biopotential signal using the plurality of subsignals, the reconstructing comprising removing the one or more signal distorting elements from the plurality of subsignals.

2. The method according to claim 1 wherein each of the plurality of electrodes comprises a contactless electrode, the contactless electrode capacitively couplable to a tissue surface of the subject.

3. The method according to claim 1 wherein the one or more signal distorting elements comprises at least one of motion artifacts, loose electrode artifacts, wandering baseline artifacts, muscle tremor artifacts, breathing artifacts, human-induced artifacts, neuromodulation artifacts, echo distortion artifacts, arterial pulse tapping artifacts and electromagnetic interference incident on at least one of the plurality of electrodes.

4. The method according to claim 1 wherein the plurality of subsignals comprises a plurality of intrinsic mode functions corresponding to the biopotential signal.

5. The method according to claim 4 wherein removing the one or more signal distorting elements from the plurality of subsignals comprises removing at least one intrinsic mode function corresponding to the one or more signal distorting elements.

6. The method according to claim 1 wherein the plurality of subsignals comprises a plurality of wavelet coefficients.

7. The method according to claim 6 wherein removing the one or more signal distorting elements from the plurality of subsignals comprises removing at least one of the plurality of wavelet coefficients corresponding to the one or more signal distorting elements.

8. The method according to claim 1 wherein the plurality of subsignals comprises a plurality of independent non-Gaussian subcomponents corresponding to the biopotential signal.

9. The method according to claim 8 wherein removing the one or more signal distorting elements from the plurality of subsignals comprises removing at least one independent non-Gaussian subcomponent corresponding to the one or more signal distorting elements.

10. The method according to claim 1 wherein identifying the one or more signal distorting elements in the plurality of subsignals comprises comparing the plurality of subsignals to one or more subsignals known to comprise at least one signal distorting element.

11. The method according to claim 1 wherein identifying the one or more signal distorting elements in the plurality of subsignals comprises comparing the plurality of subsignals to one or more threshold values known to correspond to at least one signal distorting element.

12. A system for suppressing one or more signal distorting elements from a biopotential signal indicative of a biopotential at a location on a body of a subject, the system comprising:
a plurality of electrodes couplable to a tissue surface of the subject; and
a digital signal processor, the digital signal processor configured to:
decompose the biopotential signal into a plurality of subsignals;
identify the one or more signal distorting elements in the plurality of subsignals; and
reconstruct the biopotential signal using the plurality of subsignals,
the reconstructing comprising removing the one or more signal distorting elements from the plurality of subsignals.

13. The system according to claim 12 wherein each of the plurality of electrodes comprises a contactless electrode, the contactless electrode capacitively couplable to the tissue surface of the subject.

14. A method for determining a physiological parameter based on a biopotential signal indicative of a biopotential at a location on a body of a subject, the method comprising:
acquiring the biopotential signal from the body of the subject using a plurality of electrodes;
converting the acquired biopotential signal to a digital domain;
performing a wavelet decomposition on the converted biopotential signals to generate a plurality of wavelet coefficients;
identifying a time duration between local maximum values of selected ones of the plurality of wavelet coefficients; and
extracting the physiological parameter based on the identified time duration.

15. A method according to claim 14 wherein the biopotential signal is an ECG signal and the physiological parameter is a heart rate of the subject.

16. A method according to claim 14 wherein the selected ones of the plurality of wavelet coefficients comprise wavelet coefficients from a mid-level of the wavelet decomposition.

17. A method according to claim 14 wherein identifying a time duration between local maximum values of the selected ones of the plurality of wavelet coefficients comprises identifying local maximum values which exceed a threshold value.

18. A method according to claim 17 wherein the threshold value is a percentage of a global maximum value of the selected ones of the plurality of wavelet coefficients.

19. A method according to claim 18 wherein the percentage is 60%.
